(19) United States
(12) Patent Application Publication
(10) Pub. No.: US 2023/0414802 A1
(43) Pub. Date: Dec. 28, 2023

(54) APPARATUS AND METHOD FOR SANITISING OBJECTS

(71) Applicants: John Raymond Grace, Newport Beach (AU); Clea Simes Grace, Newport Beach (AU)

(72) Inventors: John Raymond Grace, Newport Beach (AU); Clea Simes Grace, Newport Beach (AU)

(21) Appl. No.: 18/038,378

(22) PCT Filed: Nov. 23, 2021
(86) PCT No.: PCT/AU21/51394

§ 371 Date: May 23, 2023

(30) Foreign Application Priority Data
Nov. 23, 2020 (AU) 2020904325

(51) Int. Cl. A61L 2/10; H01S 3/225

(52) U.S. Cl. CPC A61L 2/10; H01S 3/225; A61L 2202/11; A61L 2202/14

(57) ABSTRACT

The present invention relates to apparatus and/or methods for sanitising objects comprising a UV light source for emitting a beam of UV light and are particularly suited to sanitising a plurality of objects such as banknotes and mail at high and variable frequencies. The apparatus and/or method may be applied to reduce a concentration of drug substance on the surface and/or to reduce microorganisms and/or viruses on the surface of each of the plurality of objects.

### CROSS-REFERENCE

This application claims priority to Australian Provisional Patent Application Number 2020904325, filed on 23 Nov. 2020, which is incorporated by reference in its entirety.

### FIELD

The present invention relates to apparatus and methods useful for sanitising objects. In particular, the apparatus and methods herein are suitable for sanitising currency such as banknotes. However, it will be appreciated that the invention is not limited to this particular field of use.

### BACKGROUND

Any discussion of the prior art throughout the specification should in no way be considered as an admission that such prior art is widely known or forms part of common general knowledge in the field.

The novel coronavirus, Severe Acute Respiratory Syndrome Coronavirus-2 (SARS-CoV-2), pandemic has escalated to unprecedented proportions since the first cases of pneumonia of unknown cause were reported to the World Health Organisation (WHO) on 31 Dec. 2019. As of 9 Oct. 2020, the virus had caused over 36 million cases of Coronavirus Disease 2019 (COVID-19) and resulted in over 1 million deaths. Cases have continued to increase rapidly worldwide in the subsequent year, with over 253 million confirmed cases of COVID-19 and over 5 million deaths reported worldwide as of 16 Nov. 2021 according to the WHO. Highly infectious and virulent novel variants of the virus have also developed.

Coronaviruses (CoVs) are enveloped, single stranded, positive sense RNA viruses from the family Coronaviridae that can cause disease in humans ranging in severity from the common cold to severe acute respiratory infections. SARS-CoV-2 can be readily transmitted between humans, resulting in exponential growth in the absence of containment measures. The precise details of how it is transmitted are not yet clear, but it is expected to spread via direct contact with an infected person or via virus-laden respiratory droplets. The droplets can be aerosolised, fall onto surfaces or be transferred directly onto surfaces when infected individuals touch them.

Once a surface has been contaminated with infectious virus, research under laboratory conditions has demonstrated it is possible for the closely related coronavirus SARS-CoV to remain infectious for up to 3-5 days when dried on paper, cloth or polymer surfaces such as PVC or Teflon. Studies of the stability on plastic surfaces have shown even longer potential durations, with low levels of SARS-CoV still able to be isolated 9-14 days after being deposited. Preliminary research indicates that SARS-CoV-2 has a similar viability and infectivity on surfaces as SARS-CoV.

This has raised concerns over the ability of everyday objects to transmit the virus from person to person and resulted in the investigation of the best methods to sanitise these objects during the ongoing pandemic. One such everyday object is currency, and in particular, banknotes. Prior to SARS-CoV-2 pandemic, it was known that many viruses and bacteria could survive for days or weeks on both paper and plastic banknotes. In laboratory settings, influenza viruses have been shown to have the capacity to survive for up to 17 days on banknotes when suspended in respiratory mucus at doses equivalent to that found in clinical specimens. The Ebola virus has also been shown to remain viable on banknotes for up to 6 days, and viable SARS-CoV-2 virus can persist on both polymer and paper banknotes for at least 21-28 days in laboratory conditions. It is therefore evident that banknotes have the capacity to harbour infectious viruses for many days after an infectious person has handled them, and once contaminated, the currency can transmit microorganisms onto the hands of an individual who touches the notes. Whilst there has been some debate over the applicability of studies of SARS-CoV-2 conducted under laboratory conditions to banknotes in circulation, a recent study of Bangladeshi banknotes showed that over 7% were contaminated with detectable levels of SARS-CoV-2 RNA. Although that study did not assess the viability of the detected virus, only the presence of its RNA, it is nonetheless evident that SARS-CoV-2 contamination of banknotes occurs frequently under real world conditions.

Processes such as cleansing with alcohol or disinfectants have been established as effective methods to sanitise surfaces to remove or inactivate pathogenic microorganisms and viruses, such as SARS-CoV-2. However, these methods are not possible for banknotes because the banknotes could be destroyed and/or rendered unsuitable for circulation. Instead, the current standard procedure for minimising the potential for contaminated banknotes to spread infection and diseases like COVID-19 is a process of quarantining, where used banknotes are removed from circulation and stored for 2-4 weeks. One disadvantage of this practice is that many more banknotes must be printed to replenish those taken out of circulation. Additionally, there is also risk that residual virus remains on the surface of the banknotes even after the resting process and thereby a risk of transmission of the virus, which leads to a further risk of infection in individuals handling the currency.

There is therefore a need for apparatus and/or methods of cleaning currency to reduce transmission of pathogenic viruses and microorganisms. In particular, there is a need for methods of cleaning currency to reduce transmission of pathogenic viruses and/or microorganisms that avoids the need to take currency out of circulation for an extended period of time.

High circulation items such as currency are also exposed to other, non-biological sources of contamination. Illicit drug substances such as cannabis, cocaine, heroin, amphetamines (e.g. methamphetamines) and ecstasy (MDMA) are reportedly present on up to or greater than 90% of Australian, American, and Canadian banknotes (e.g., Amanda J Jenkins (2001) Forensic Science International, 121(3), 189-193). Many other countries around the world also have currency in circulation contaminated with illicit drug residues (e.g., Troiano et al., (2017) European Journal of Public Health, 27(6), 1097-1101). Cocaine in particular is commonly found on banknotes throughout the world and is thought to be more prevalent than other drug substances because it is more resistant to degradation. The amount of illicit drug substance per banknote can vary from nanogram quantities to tens or hundreds of micrograms or more and is generally transferred to banknotes during illicit drug trade. However, existing banknote sorting, counting and stacking processes can transfer drug substances from banknote to banknote, acting as a source of contamination for new banknotes. As the presence of illicit substances on banknotes can be detected and potentially used as evidence of criminal activity, and as drug substances can be ingested by people, including children, through touching contaminated banknote surfaces, there is a need for methods of cleaning currency to reduce the concentration of illicit drug substances thereon.

It is an object of the present invention to overcome or ameliorate at least one of the disadvantages of the prior art, or to provide a useful alternative.

### SUMMARY

The present invention relates to apparatus and/or methods for sanitising objects. According to a first aspect of the invention there is provided an apparatus for sanitising a surface of an object, comprising: a UV light source for emitting a beam of UV light; and a system for conveying the object through the beam; wherein the UV light has a wavelength, intensity and pulse duration sufficient to reduce microorganisms and/or viruses on the surface.

The following features may be used alone or in combination with the first aspect above: The UV light source may be a laser. The laser may be an excimer laser.

According to a second aspect of the invention there is provided an apparatus for sanitising a surface of an object, comprising: a UV light source for emitting a beam of UV light, wherein the UV light source is an excimer laser; and a system for conveying the object through the beam; wherein the UV light has a wavelength, intensity and pulse duration sufficient to reduce microorganisms and/or viruses on the surface.

The following features may be used alone or in combination with the first or second aspects above: The UV light may have a wavelength, intensity and pulse duration sufficient to reduce viable microorganisms and/or viruses on the surface. The UV light may have a wavelength, intensity and pulse duration sufficient to inactivate microorganisms and/or viruses on the surface. The apparatus may further comprise a turning mirror for directing the beam of UV light towards the object. The apparatus may further comprise a beam expansion lens or beam expanding telescope for controlling beam size. The apparatus may further comprise a trigger system configured to detect the presence of the object and command the UV light source to emit a beam of UV light. The apparatus may further comprise a polarising element for polarising the beam of UV light. The apparatus may further comprise a flipping mirror for directing alternate pulses of the laser into two separate beam paths. The flipping mirror may be a single axis galvanometer mirror. The flipping mirror may be a 2-dimensional galvanometer mirror or mirror set. The apparatus may further comprise a retroreflective mirror for reflecting the beam of UV light onto the object. The apparatus may further comprise a beam splitting element for splitting the beam of UV light.

According to a third aspect of the invention there is provided a method for sanitising a surface of an object, comprising: irradiating the object with UV light, wherein the UV light is emitted by a laser and has a wavelength, intensity and pulse duration sufficient to reduce microorganisms and/or viruses on the surface. In the third aspect, the laser may be an excimer laser.

According to a fourth aspect of the invention there is provided a method for sanitising a surface of an object, comprising: irradiating the object with UV light, wherein the UV light is emitted by an excimer laser and has a wavelength, intensity and pulse duration sufficient to reduce microorganisms and/or viruses on the surface.

The following features may be used alone or in combination with the third or fourth aspects above: The UV light may have a wavelength, intensity and pulse duration sufficient to reduce viable microorganisms and/or viruses on the surface. The UV light may have a wavelength, intensity and pulse duration sufficient to inactivate microorganisms and/or viruses on the surface. The wavelength may be 193 nm or the wavelength may be 248 nm. The intensity may be greater than 6,000 W/cm². The intensity may be greater than 500,000 W/cm². The pulse duration may be from about 5 to about 35 ns. The energy incident on the surface from a single pulse may be at least 0.1 mJ/cm². The energy incident on the surface from a single pulse may be at least 2.5 mJ/cm². The UV light may be focussed on an area corresponding to the size and shape of the surface of the object. The method may comprise irradiating the object with a single pulse of UV light from a single light source. The method may comprise irradiating the object with two pulses of UV light from a single light source. The light source may illuminate the surface of the object uniformly or substantially uniformly. The method may comprise: irradiating the object with a first pulse of UV light from a first light source, and subsequently irradiating the object with a second pulse of UV light from a second light source. The surface may comprise a polymer. The object may be a banknote. There may be a 2 log 10 reduction in the number of microorganisms and viruses on the surface. The object may be moving at a speed of 1.0 m/s with respect to the laser. A plurality of objects may be moving at a speed with respect to the laser that results in a frequency of between 10 Hz and 50 Hz with respect to the laser. In some embodiments, a plurality of objects may be moving at a speed with respect to the laser that results in a frequency of between 5 Hz and 50 Hz with respect to the laser. The microorganisms and/or viruses may comprise SARS-CoV-2.

According to a fifth aspect of the invention there is provided a sanitised object produced by the method of the third or fourth aspect above.

According to a sixth aspect of the invention there is provided a method according to the third or fourth aspect above utilising the apparatus according to the first or second aspect above.

According to a seventh aspect of the invention there is provided an apparatus according to the first or second aspect above when utilised in the method according to the third or fourth aspect above.

According to an eighth aspect of the invention there is provided a method for sanitising a surface of an object, comprising: conveying the object through UV light from an excimer laser at a speed of greater than 1.0 m/s, such that the UV light irradiates the surface, wherein the UV light has a wavelength, intensity and pulse duration sufficient to reduce a concentration of drug substance on the surface.

According to a ninth aspect of the invention there is provided a method for sanitising a surface of an object, comprising: conveying the object through UV light from an excimer laser at a speed of greater than 1.0 m/s, such that the UV light irradiates the surface, wherein the UV light has a wavelength, intensity and pulse duration sufficient to reduce microorganisms and/or viruses on the surface.

The following features may be used alone or in combination with the eighth and/or ninth aspects above: The method may be for sanitising a surface of each of a plurality of objects, and comprise conveying the objects through UV light from an excimer laser at a speed of greater than 1.0 m/s and at a frequency of greater than 10 Hz, such that the UV light irradiates the surface of each of the plurality of objects, wherein the UV light has a wavelength, intensity and pulse duration sufficient to reduce a concentration of drug substance on the surface of each of the plurality of objects. The objects may be conveyed through the UV light at a frequency of between 10 Hz and 50 Hz. The UV light may have a wavelength, intensity and pulse duration sufficient to chemically alter or destroy the drug substance on the surface. The drug substance may be cocaine. The method may be for sanitising a surface of each of a plurality of objects, and comprise conveying the objects through UV light from an excimer laser at a speed of greater than 1.0 m/s and at a frequency of greater than 5 Hz or greater than 10 Hz, such that the UV light irradiates the surface of each of the plurality of objects, wherein the UV light has a wavelength, intensity and pulse duration sufficient to reduce microorganisms and/or viruses on the surface of each of the plurality of objects. The objects may be conveyed through the UV light at a frequency of between 5 Hz and 50 Hz or between 10 Hz and 50 Hz. The UV light may have a wavelength, intensity and pulse duration sufficient to inactivate microorganisms and/or viruses on the surface. The microorganisms and/or viruses may comprise SARS-CoV-2. There may be a 2 log 10 reduction in the number of microorganisms and viruses on the surface. The UV light may irradiate the surface for a period of from 5 ns to 35 ns. The wavelength may be 193 nm or the wavelength may be 248 nm. The intensity may be greater than 6,000 W/cm². The intensity may be greater than 500,000 W/cm². The pulse duration may be from about 5 to about 35 ns. A single pulse of the UV light may have an energy of at least 0.1 mJ/cm² on the surface. A single pulse of the UV light may have an energy of at least 1.0 mJ/cm² on the surface. A single pulse of the UV light may have an energy of at least 2.5 mJ/cm² on the surface. A single pulse of the UV light may have an energy of at least 4.0 mJ/cm² on the surface. The UV light may have a focal spot size approximately the same size as the or each object. The method may comprise irradiating the or each object with a single pulse of UV light from a single excimer laser. The method may comprise irradiating the or each object with two pulses of UV light from a single excimer laser. The method may comprise irritating the or each object with 2, 4, 6 or 12 pulses of UV light from a single excimer laser. The number of pulses of UV light from a single excimer laser will depend on the number of pulses per second the laser generates and the number of objects per second that are irradiated. A single pulse of UV light may illuminate the surface of the or each object uniformly or substantially uniformly. The method may comprise: irradiating a first surface of the or each object with a first pulse of UV light from a first excimer laser, and subsequently irradiating a second surface of the or each object with a second pulse of UV light from a second excimer laser. The first surface and the second surface of the or each object may partially or completely overlap. The first surface and the second surface of the or each object may not overlap. The first pulse and the second pulse may be delivered sequentially. The first pulse and the second pulse may be delivered simultaneously. The or each object may be conveyed through the UV light at a speed of between 1.0 m/s and 8.0 m/s. The UV light may be directed towards the or each object with a turning mirror. The UV light may be controlled with a beam expansion lens or beam expanding telescope. The excimer laser may be fitted with an unstable resonator. Presence of the or each object may be detected by a trigger system configured to command the excimer laser to emit the UV light. The UV light may be polarised using a polarising element. A flipping mirror may be used to direct alternate pulses of UV light into two separate beam paths. The flipping mirror may be a single axis galvanometer mirror, a 2-dimensional galvanometer mirror or mirror set, or rotating polygon that may be synchronised to the product speed. The method may comprise reflecting part or all of the UV light onto the or each object using a retroreflective mirror. The UV light may be split using a beam splitting element. The surface may comprise a polymer. The or each object may be a banknote.

According to a tenth aspect of the invention there is provided a sanitised object produced by the method of the eighth and/or ninth aspects above.

According to an eleventh aspect of the invention there is provided an apparatus for sanitising a surface of an object, comprising: an excimer laser that emits pulses of UV light; and a system that conveys the object(s) through the UV light at a speed of greater than 1.0 m/s, optionally between 1.0 m/s and 8.0 m/s; wherein the UV light has a wavelength, intensity and pulse duration sufficient to reduce a concentration of a drug substance on the surface and/or reduce microorganisms and/or viruses on the surface.

The following features may be used alone or in combination with the eleventh aspect above: The apparatus may comprise: an excimer laser that emits pulses of UV light; and a system that conveys the object(s) through the UV light at a speed of greater than 1.0 m/s, optionally between 1.0 m/s and 8.0 m/s; wherein the UV light has a wavelength, intensity and pulse duration sufficient to reduce a concentration of a drug substance on the surface and/or reduce microorganisms and/or viruses on the surface. The drug substance may be cocaine. The UV light may have a wavelength, intensity and pulse duration sufficient to inactivate microorganisms and/or viruses on the surface. A single pulse of the UV light may have an energy of at least 4 mJ/cm² on the surface. The apparatus may further comprise any one or more of the following: a turning mirror for directing the UV light towards the object; a beam expansion lens or beam expanding telescope for controlling beam size; a trigger system configured to detect the presence of the object and command the UV light source to emit pulses of UV light a polarising element for polarising the UV light; a flipping mirror for directing alternate pulses of the laser into two separate beam paths; a retroreflective mirror for reflecting the UV light onto the object; or a beam splitting element for splitting the UV light. The apparatus may further comprise a turning mirror for directing the UV light towards the object. The apparatus may further comprise a beam expansion lens or beam expanding telescope for controlling beam size. The apparatus may further comprise a trigger system configured to detect the presence of the object and command the UV light source to emit a beam of UV light. The apparatus may further comprise a polarising element for polarising the UV light. The apparatus may further comprise a flipping mirror for directing alternate pulses of the laser into two separate beam paths. The flipping mirror may be a single axis galvanometer mirror. The flipping mirror may be a 2-dimensional galvanometer mirror or mirror set. The apparatus may further comprise a retroreflective mirror for reflecting the UV light onto the object. The apparatus may further comprise a beam splitting element for splitting the UV light. The excimer laser may be fitted with an unstable resonator.

According to a twelfth aspect of the invention there is provided the method of the eighth and/or ninth aspects above utilising the apparatus of the eleventh aspect above.

According to a thirteenth aspect of the invention there is provided the apparatus of the eleventh aspect above when utilised in the method of the eighth and/or ninth aspects above.

### BRIEF DESCRIPTION OF THE DRAWINGS

Embodiments of the invention will now be described, by way of example only, with reference to the accompanying drawings in which:

FIG. 1 is a diagrammatic representation of a side view of an apparatus according to an embodiment of the present invention, where a laser source emits a UV laser beam that is directed onto a banknote using a turning mirror.

FIG. 2 is a diagrammatic representation of a side view of an apparatus according to an embodiment of the present invention, where a laser source emits a UV laser beam that is directed in part onto a banknote using a turning mirror and in part onto a retroreflecting mirror that reflects that part of the incident beam onto the underside of the banknote.

FIG. 3 is a diagrammatic representation of a side view of an apparatus according to an embodiment of the present invention, where a laser source emits a UV laser beam whose pulses are alternately directed into two beam paths by a flipping mirror, where the first pulse is directed, using a first turning mirror, onto a banknote at a first position, and the second pulse is directed, using a second turning mirror, onto a banknote at a second position spaced apart from the first position.

FIG. 4 is a diagrammatic representation of a side view of an apparatus according to an embodiment of the present invention, where a laser source emits a UV laser beam that is split into two polarised beam components, the S component and the P component, by a polarising mirror, where both beam components can be separately directed onto the banknote.

FIG. 5 is a diagrammatic representation of a side view of an apparatus according to an embodiment of the present invention, where a laser source emits a UV laser beam that is directed onto a variety of objects through use of one or more scanning mirror(s).

FIG. 6 is a diagrammatic representation of a side view of an apparatus according to an embodiment of the present invention, where a first laser source emits a first UV laser beam and a second laser source emits a second UV laser beam, wherein the first and second laser beam pulses are alternately directed into two beam paths by a flipping mirror, the first pulse being directed onto a banknote at a first position, and the second pulse being directed onto a banknote at a second position spaced apart from the first position.

FIG. 7 is a diagrammatic representation of a side view of an apparatus according to an embodiment of the present invention, where two separate laser sources each emit a UV laser beam that is directed onto a banknote using a turning mirror.

FIG. 8 is a representative image of spot titre agar plates enumerating (i) Lambda bacteriophage plaques forming units and (ii) E. coli colony forming units 24 h post 248 nm sanitising.

FIG. 9 is summarised data of the viable virus levels remaining after sanitising with an excimer laser at 193 nm over various starting viral contamination levels.

FIG. 10 is summarised data of the viable virus levels remaining after sanitising with an excimer laser at 248 nm over various starting viral contamination levels.

FIG. 11 is summarised data of the viable bacteria levels remaining after sanitising with an excimer laser at 248 nm over various starting bacterial contamination levels.

FIG. 12 is summarised data of the viable bacteria levels remaining after sanitising with an excimer laser at 193 nm over various starting bacterial contamination levels.

FIG. 13 shows the correlation between the median colony forming units (CFU) assessed in this study and the levels of contamination found on notes in circulation summarised from Vriesekoop et al.

FIG. 14 shows data for sanitising E. coli contamination on plastic surfaces. Statistical analysis has been conducted as described in the text-, *** p<0.001.

FIG. 15 shows photographs of undamaged Australian banknotes post laser treatment at 248 nm with 4-4000 mJ/cm². Each treated region has been matched with a nearby untreated (0 mJ/cm²) region located on the same banknote for comparison. The entire area of the treated samples shown was treated (˜7×7 mm; ˜7×5 mm of which is shown) and there is no perceptible damage, even at ˜40× magnification.

FIG. 16 shows standard concentration vs. absorbance curves pooled from all experiments and linear regression analyses for standardised (i) HBB and (ii) DEX reactions with KMnO₄ under alkaline conditions.

FIG. 17 shows the level of DEX remaining post 193 nm laser treatment on (i) plastic (polystyrene) petri dishes and (ii) paired analyses on banknotes. The key for interpretation of FIG. 17(i) is the same as that shown in FIG. 18.

FIG. 18 shows the level of DEX remaining post 193 nm laser treatment on banknotes: (i) Unpaired (HBB1) and paired (HBB2) analyses of HBB sanitising; (ii) Depiction of the relationship between drug contamination in control and 5 mJ/cm² data with the pairs of samples shown.

FIG. 19 shows the level of HBB) and DEX remaining post 248 nm laser treatment on contaminated banknotes.

FIG. 20 shows representative images of a saliva detection test of amphetamine after laser treatment of DEX-contaminated banknotes. The scheme for reading the results is shown on the right-hand side.

### DEFINITIONS

In describing and claiming the present invention, the following terminology will be used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of ‘including, but not limited to’.

Other than in the operating examples, or where otherwise indicated, all numbers expressing quantities of ingredients or reaction conditions used herein are to be understood as modified in all instances by the term ‘about’.

In what follows, unless otherwise indicated, ‘%’ will mean ‘weight %’ and ‘ratio’ will mean ‘weight ratio’. As used herein, the term “weight %” or “wt %” is calculated as [100×mₓ/mₜₒₜ], where mₓ is the mass of component x and mₜₒₜ is the total mass of all components.

The term ‘substantially’ as used herein shall mean comprising more than 50% by weight, where relevant, unless otherwise indicated.

The recitation of a numerical range using endpoints includes the endpoints and all numbers subsumed within that range (e.g., from 1 to 10 includes 1, 1.5, 2, 5, 5.75, 7.1, 8.5, 9, 10, etc.). The same understanding applies to a numerical range using endpoints and the term “between” and includes the endpoints (e.g., from between 1 to 10 includes 1, 1.5, 2, 5, 5.75, 7.1, 8.5, 9, 10, etc.).

As used herein and in the claims, the singular form of “a”, “an”, and “the” may include the plural referents unless the context clearly dictates otherwise. By way of example, references herein to a singular “object” may include the plural referents “objects” or “plurality of objects” unless the context clearly dictates otherwise.

As used herein, the terms “about” and “approximately” are used synonymously. Both terms are meant to cover any normal fluctuations or variations understood by those skilled in the art. In some embodiments, “about” and “approximately” refer to ±10% of the reference value, or ±5%, or ±2%, or ±1%, or ±0.1% of the reference value.

In this application, the use of “or” means “and/or” unless stated otherwise. The use of “A and/or B” is used to indicate that in one embodiment, components A and B may be present, or that in another embodiment, that component A may be present, or that in another embodiment that component B may be present.

As used herein, unless the context clearly indicates otherwise, the terms “light”, “light beam”, “beam”, “pulse”, “artificial radiation”, “radiation” and the like all refer to UV light, or a UV light beam. It will be understood that the UV light, and particularly UV light emitted by a laser, travels in beams, and accordingly, any reference herein to a “UV light beam” or “beam” in the context of UV light from a laser is also a reference to “UV light” (and vice versa).

As used herein, the terms “illuminate” and “irradiate” and related terms such as “illuminating”, “illuminated”, “irradiating”, and “irradiated” in the context of an object or surface thereof refers to receiving laser UV light and in the context of a laser beam or laser beam source refers to emitting laser UV light, unless the context clearly indicates otherwise.

### DETAILED DESCRIPTION

The skilled addressee will understand that the invention comprises the embodiments and features disclosed herein as well as all combinations and/or permutations of the disclosed embodiments and features.

### Object, UV Light and Light Source

Objects capable of being sanitised by the apparatus and/or methods herein are not particularly limited. However, the apparatus and/or methods herein are particularly suited to sanitising objects that require sorting, and more particularly, objects that require sorting at a frequency of greater than 1 Hz. In some embodiments, the apparatus and/or methods herein are particularly suited to sanitising objects that are singularised and separated. The singularising and separating may be a standard part of processing or sorting of the object. In other embodiments, the apparatus and/or methods herein are particularly suited to sanitising objects that are moving. The movement may be a standard part of the processing or sorting of the object. As used herein, the frequency “x Hz” in the context of a moving object refers to the object moving past a fixed point at a rate of x objects per second. In some embodiments, the fixed point is the UV light source or laser. In other embodiments, another fixed point may be chosen as a reference point for measuring the frequency at which an object is passing through the UV light beam, such as the point where the UV light beam is incident on an illumination stage. Accordingly, a range of objects including currency, such as coins and/or banknotes, or mail, such as parcels and/or letters, or goods, such as boxes, packages, containers and/or bottles are particularly suitable for sanitising using the apparatus and/or methods described herein.

In some embodiments, the apparatus and/or methods herein are particularly suited to sanitising objects that are moving or conveyed at a frequency of greater than 2 Hz, greater than 3 Hz, greater than 4 Hz, greater than 5 Hz, greater than 6 Hz, greater than 7 Hz, greater than 8 Hz, greater than 9 Hz, greater than 10 Hz, greater than 20 Hz, 30 Hz, greater than 40 Hz or greater than 50 Hz, or of a frequency between 1 Hz and 50 Hz, or of between 1 Hz and 10 Hz, or of between 10 Hz and 25 Hz, or of between 10 Hz and 50 Hz, or of between 20 Hz and 50 Hz, or of between 1 Hz and 5 Hz, or of between 30 Hz and 45 Hz, or of frequencies of about 1 Hz, 2 Hz, 3 Hz, 4 Hz, 5 Hz, 6 Hz, 7 Hz, 8 Hz, 9 Hz, 10 Hz, 15 Hz, 20 Hz, 20 Hz, 25 Hz, 30 Hz, 35 Hz, 40 Hz, 45 Hz, or 50 Hz with respect to the UV light source or laser. It will be understood that such embodiments necessarily require that a plurality of objects pass through the UV light beam. As used herein, the term “plurality” refers to two or more objects. In some embodiments, the term “plurality” refers to at least 2, at least 5, at least 10, at least 50, at least 100, at least 250, at least 500, at least 1000, at least 5000, or at least 10000 or more objects, or to 2, 5, 10, 50, 100, 250, 500, 1000, 5000, 10000 or more objects. In some embodiments, the apparatus and/or methods herein are particularly suited to sanitising objects that are conveyed at a frequency of greater than 5 Hz. In some embodiments, the apparatus and/or methods herein are particularly suited to sanitising objects that are conveyed at a frequency of between 5 Hz and 50 Hz. In some embodiments, the apparatus and/or methods herein are particularly suited to sanitising objects that are conveyed at a frequency of greater than 10 Hz. In some embodiments, the apparatus and/or methods herein are particularly suited to sanitising objects that are conveyed at a frequency of between 10 Hz and 50 Hz. It will be understood that in such embodiments, the conveying need reach frequencies of greater than 10 Hz for at least part of the duration of sanitising but need not be at frequencies of greater than 10 Hz for the entire duration of sanitising. In such embodiments, this is because the conveying may be staged, comprising a start-up stage, an operational stage, and/or a shut down stage, throughout which the frequency of conveying may change as described below.

In some embodiments, the apparatus and/or methods herein are particularly suited to sanitising objects that are moving or conveyed at a speed of greater than 0.1 m/s, greater than 0.2 m/s, greater than 0.3 m/s, greater than 0.5 m/s, greater than 0.75 m/s, greater than 1.0 m/s, greater than 1.5 m/s, greater than 2.0 m/s, greater than 2.5 m/s, greater than 3.0 m/s, greater than 3.5 m/s, greater than 4.0 m/s, greater than 4.5 m/s, greater than 5.0 m/s, greater than 5.5 m/s, greater than 6.0 m/s, greater than 6.5 m/s, greater than 7.0 m/s, greater than 7.5 m/s, greater than 8.0 m/s, or of a speed between 0.1 m/s and 8.0 m/s, or of between 0.1 m/s and 1.0 m/s, or of between 1.0 m/s and 3.0 m/s, or of between 2.0 m/s and 6.0 m/s, or of between 3.0 m/s and 8.0 m/s, or of between 6.0 m/s and 8.0 m/s. As used herein, the speed “x m/s” in the context of a moving object refers to the object moving past a fixed point at a rate of x metres per second. In some embodiments, the fixed point is the UV light source or laser. In other embodiments, another fixed point may be chosen as a reference point for measuring the speed at which an object is passing through the UV light beam, such as the point where the UV light beam is incident on an illumination stage. It will be understood that a single object may be moved at these speeds in these embodiments. It will also be understood that a plurality of objects may be moved at these speeds in these embodiments. In some embodiments, all objects may be moving at the same speed. In some embodiments, the apparatus and/or methods herein are particularly suited to sanitising objects that are conveyed at a speed of greater than 1.0 m/s. In some embodiments, the apparatus and/or methods herein are particularly suited to sanitising objects that are conveyed at a speed of between 1.0 m/s and 8.0 m/s. It will be understood that in such embodiments, the conveying need reach speed of greater than 1.0 m/s for at least part of the duration of sanitising but need not be at speeds of greater than 1.0 m/s for the entire duration of sanitising. In such embodiments, this is because the conveying may be staged, comprising a start-up stage, an operational stage, and/or a shut down stage, throughout which the speed of conveying may change as described below.

The object herein may comprise one or more surfaces. The apparatus and/or methods herein target a surface of the object and sanitise that surface. The surface has a composition that is not particularly limited. In some embodiments, the surface comprises a polymer. In some embodiments, the polymer is polypropylene. In some embodiments, the surface comprises a wood pulp-based material. In some embodiments, the surface comprises paper. In some embodiments, the surface comprises cardboard. In some embodiments, the surface comprises a textile. In some embodiments, the surface comprises a natural fibre-based textile. In some embodiments, the surface comprises a synthetic fibre-based textile. In some embodiments, the surface comprises cotton. In some embodiments, the surface comprises cotton paper. Cotton paper may include cotton fibres or may include cotton fibres mixed with one or more other textile fibres including linen or abaca. In some embodiments, the surface comprises a metal. In some embodiments, the surface comprises an alloy. In some embodiments, the surface comprises glass. In some embodiments, the surface comprises one or more materials selected from: polymer, paper, cardboard, synthetic textile, natural textile, glass, metal, and alloy. The object sanitised herein is not particularly limited. In some embodiments, the object is a solid object. The solid object may take any suitable form or shape, including cubes, rectangular prisms, flat sheets, and the like. In other embodiments, the object is hollow. The hollow object may take any suitable form or shape, including a box, a carton, a bottle, and the like. In such embodiments, the surface sanitised by the methods herein may be an outer-facing surface. In some embodiments, the apparatus and/or methods described herein sanitise a single object at a time. In such embodiments, the apparatus and/or methods described herein may sanitise a single surface of a single object at a time or may sanitise two surfaces of a single object at the same time. In some embodiments, the apparatus and/or methods described herein sanitise a single object per laser pulse. In such embodiments, the apparatus and/or methods described herein may sanitise a single surface of a single object per laser pulse. In other embodiments, the apparatus and/or methods described herein may sanitise two surfaces of a single object per laser pulse. In some embodiments, the apparatus and/or methods described herein sanitise two or more objects per laser pulse. In such embodiments, the apparatus and/or methods described herein may sanitise one surface of each of two or more objects per laser pulse. In other embodiments, the apparatus and/or methods described herein may sanitise two or more surfaces of each of two or more objects per laser pulse. In some embodiments, multiple laser pulses may be used to sanitise a single surface of a single object. In some embodiments, multiple laser pulses may be used to sanitise two or more surfaces of a single object. In some embodiments, multiple laser pulses may be used to sanitise two or more surfaces of two or more objects.

In some embodiments the apparatus and/or methods herein target two or more surfaces of an object and sanitise each of those surfaces. In some embodiments, the apparatus and/or methods herein target and sanitise all surfaces of an object. In some embodiments, the apparatus and/or methods herein sanitise all surfaces of an object simultaneously. In some embodiments, the apparatus and/or methods herein sanitise each surface of an object separately. In some embodiments, the apparatus and/or methods herein sanitise a surface of an object in full. In some embodiments, the apparatus and/or methods herein sanitise a surface of an object in part. In some embodiments, the surface of the object is flat or substantially flat. In other embodiments, the surface of the object is curved or substantially curved. The UV light is advantageously adapted to irradiate one or more flat surfaces of an object, or one or more curved surfaces of an object, or one or more complex surfaces of an object comprising flat region(s) and curved region(s). The term “complex surface” as used herein refers to a surface comprising one or more flat or substantially flat regions and one or more curved or substantially curved regions. As will be apparent, the form of the surface of the object is not particularly limited. The object may have a surface that is smooth. The object may have a surface that is textured. In certain embodiments, a textured surface may require a higher intensity, greater pulse duration, greater pulse energy, or greater number of pulses for a given wavelength compared to a smooth surface to achieve inactivation of microorganisms and/or viruses. The surface may be dry or substantially dry.

In one embodiment, the apparatus and/or methods herein are particularly suited to sanitising currency, and more specifically, banknotes. Banknotes have a high surface area to volume ratio, generally having face dimensions of height between about 6 cm and about 8 cm and width of between about 13 cm and about 16 cm, and having a depth of between about 0.1 mm and 0.3 mm. They can be composed of polymer, such as polypropylene, or may be paper based, composed of cotton paper.

Banknotes may comprise three-dimensional security features, including but not limited to embossed features or protruding shapes or sections that add texture to the banknote surface. In some embodiments, texture on the banknote surface may arise from application of ink. In some embodiments, the banknotes comprise one or more grooves. In use, banknotes may become physically deformed or creased, such that in some embodiments, used banknotes have greater three-dimensionality of shape than newly manufactured banknotes. The present inventors have discovered that precise doses of UV radiation can be delivered to sanitise banknote surfaces during the sorting process using embodiments of the apparatus and/or methods described herein such that variations in surface texture or profile (such as might result from physical deformity of banknotes as a result of use) can be overcome. Additionally, although UV light from lasers is coherent (compared to UV lamp sources, which emit incoherent light) and is therefore more sensitive to surface texture than light from lamp sources, the present inventors have shown that embodiments of the apparatus and/or methods described herein utilising a single beam sanitise banknote surfaces despite the presence of three-dimensional security features thereon. However, in some embodiments, the apparatus and/or methods described herein can be utilised to emit one or more beams of UV light at different and adjustable incident angles such that textural variations of other banknotes or objects with three-dimensional surface texture do not present a barrier during the UV sanitising process.

Banknotes or parts thereof may also comprise one or more layers of ink. In some embodiments, the ink is printed on a polymer substrate, such that the banknote has an outermost surface comprising ink. In other embodiments, the ink is printed on a paper substrate, wherein the ink is partially or completely absorbed in the paper substrate layer. Embodiments of the apparatus and/or methods described herein are advantageously effective in sanitising ink printed polymer and paper banknotes. Without wishing to be bound by theory, ink in or on the banknote surface may increase the UV susceptibility of the banknote, and/or may increase heat sensitivity relative to a banknote without ink. Accordingly, the apparatus and/or methods described herein are advantageously capable of sanitising the banknotes in a rapid manner whilst minimising the cumulative UV radiation exposure time for each individual banknote.

Although three-dimensional texture and/or ink may be features of banknotes, it is to be understood that the apparatus and/or methods described herein are not limited to sanitising banknotes. In other embodiments, other objects may also be sanitised, and these objects may have three-dimensional texture and/or ink in or on a surface thereof and therefore benefit from the advantages of the apparatus and/or methods described herein as noted above in relation to banknotes.

Banknotes are processed using sorting machines that generally singularise notes at frequencies of between about 10 Hz and 50 Hz, with many singularising notes at a frequency of between 10 Hz and 20 Hz. In some embodiments, the apparatus and/or methods herein advantageously sanitise banknotes in situ during sorting without the need to reduce the frequency of the singularisation or sorting process to below 30 Hz, or below 25 Hz, or below 20 Hz, or below 15 Hz, or below 10 Hz, or below 5 Hz, or below 4 Hz, or below 3 Hz, or below 2 Hz, or below 1 Hz, or to 0 Hz. In some embodiments, this is achieved through use of UV light having a pulse duration that is short enough to irradiate a banknote surface with a full pulse in an effectively frozen position despite the banknote moving past the laser beam. In some embodiments, sorting and processing speeds can exceed 3 m/s and at these speeds, a short laser pulse in the order of about 5 to about 35 ns may effectively “freeze” the banknote and fully illuminate it as it passes by.

The apparatus and/or methods herein utilise ultra-violet (UV) radiation to sanitise objects. As used herein, the term “UV radiation” is to be understood as synonymous with “UV light”, and the term “radiation” is to be understood as synonymous with the term “light”. The radiation may have any suitable wavelength, but in some embodiments, is UV-C radiation having a wavelength of from about 100 nm to about 280 nm. In one embodiment, the UV-C radiation has a wavelength of from about 100 nm to about 150 nm, or of from about 150 nm to about 200 nm, or of from about 180 nm to about 200 nm, or of from about 200 nm to about 250 nm, or of from about 210 nm to about 280 nm, or of from about 180 nm to about 230 nm. In some embodiments, the radiation has a wavelength of less than about 250 nm, or less than about 230 nm, or less than about 210 nm, or less than about 200 nm. In other embodiments, the UV-C radiation has a wavelength of about 100 nm, about 120 nm, about 140 nm, about 160 nm, about 180 nm, about 200 nm, about 220 nm, about 240 nm, about 260 nm, or about 280 nm. In one embodiment, the UV-C radiation has a wavelength of 193 nm. In one embodiment, the UV-C radiation has a wavelength of 248 nm. In other embodiments, UV-B radiation may be used. In such embodiments, the UV-B radiation may have a wavelength of from about 280 nm to about 315 nm, or of from about 280 nm to about 290 nm, or of from about 290 nm to about 300 nm, or of from about 295 nm to about 305 nm, or of from about 300 nm to about 310 nm, or of from about 295 nm to about 315 nm. In other embodiments, the UV-B radiation has a wavelength of about 280 nm, about 285 nm, about 290 nm, about 295 nm, about 300 nm, about 305 nm, about 310 nm, or about 315 nm. In one embodiment, the UV-B radiation has a wavelength of 308 nm. In yet further embodiments, the UV radiation is UV-A radiation. In some embodiments, the UV-A radiation has a wavelength of from about 315 nm to about 400 nm, or from about 315 nm to about 350 nm, or from about 325 nm to about 375 nm, or from about 350 nm to about 400 nm, or of about 315 nm, 320 nm, 325 nm, 330 nm, 335 nm, 340 nm, 345 nm, 350 nm, 351 nm, 360 nm, 370 nm, 380 nm, 390 nm, or 400 nm.

The UV radiation may originate from an artificial radiation source. In some embodiments, the artificial radiation source is a laser. Accordingly, the UV light source as described herein may be a laser. The laser may be an excimer or “excited dimer” laser. Excimer lasers advantageously emit UV radiation of a higher intensity than that emitted by UV lamps. Accordingly, the UV light source as described herein may be an excimer laser. The excimer laser may be any suitable excimer laser, in some embodiments being selected from the group consisting of an ArF, KrCl, KrF, XeBr, XeF and an XeCl excimer laser. In one embodiment, the laser is an excimer laser selected from the group consisting of ArF, KrF or XeCl. In one embodiment, the laser is an excimer laser selected from ArF (193 nm) or KrF (248 nm). In one embodiment, the wavelength emitted by the UV light source is 193 nm or 248 nm. In another embodiment, the wavelength emitted by the excimer laser UV light source is 193 nm or 248 nm. The excimer laser may be an ArF laser (193 nm). The excimer laser may be a KrCl laser (222 nm). The excimer laser may be a KrF laser (248 nm). The excimer laser may be a XeBr laser (282 nm). The excimer laser may be an XeCl laser (308 nm). The excimer laser may be an XeF laser (351 nm). The excimer laser may be a 100 W class laser. In one embodiment, the excimer laser is a KrF laser having an energy of 500 mJ at 200 Hz. Other lasers suitable for use in the present invention may include ArF (200 mJ), KrF (500 mJ), XeCl (350 mJ), and XeF (300 mJ). In some embodiments, these lasers are run at between approximately 50% and 100% of the specified energies. In some embodiments, if lower energies are needed, an attenuator may be used, permitting a stable range of operation as low as about 1% of the specified energies, or of between approximately 10% and 100% of the specified energies. Other classes of laser include 300 W, 600 W classes, and 1200 W, and these may be suitable for use herein in some embodiments.

The repetition rate flexibility of excimer lasers is a property that makes them particularly suited to the apparatus and/or methods described herein. Repetition rate flexibility allows excimer lasers to switch frequencies and hence they may be used continuously as an object conveyer system ramps up to full speed from stop. In other words, embodiments herein where an excimer laser is used advantageously enable the apparatus and/or methods described herein to be retrofitted to existing sorting machines, including banknote sorting machines, where the machines may convey objects to be sanitised at a non-uniform speed and/or frequency. By way of non-limiting example, a banknote sorting machine may operate with a speed that varies between a start up speed, an operational speed, and a shut down speed. Start up speeds may speed up from 0 m/s to from 1 to 8 m/s. Operational speeds may be in the range 1 to 8 m/s. Shut down frequencies may slow down from between 1 and 8 m/s to 0 m/s. In some embodiments, a banknote sorting machine may operate with a frequency that vanes between a start up frequency, an operational frequency, and a shut down frequency. Start up frequencies may speed up from 0 Hz to from 10 Hz to 50 Hz. Operational frequencies may be in the range 10 Hz to 50 Hz. Shut down frequencies may slow down from between 10 Hz and 50 Hz to 0 Hz. It may be desirable for banknote sorting bodies such as banks to sanitise banknotes during all stages of the sorting operation, including start up and shut down. The present inventors have discovered that the feature of excimer lasers of variability in repetition rate enables the same apparatus and/or methods to sanitise objects conveyed past the excimer laser UV beam at variable speed. Retrofit of the apparatus and/or method of sanitising objects described herein to current banknote sorting machinery may avoid costly modification of currently used equipment. It will also be understood that the retrofit capabilities of the apparatus and/or method of sanitising objects described herein may apply more broadly to other (non-banknote) systems where objects are moved at speed, such as postal sorting machinery.

It is envisioned that other (non-excimer) lasers may be useful in other embodiments, such as where an object conveyer system is continuous or where objects are not presented to the light source until a stable speed has been reached. In such embodiments, however, it will be understood that the non-excimer lasers are unable to switch frequencies when or if the conveyer system speed changes.

The laser may emit radiation having any suitable intensity. In one embodiment, the intensity or field strength of the UV radiation emitted by the laser is greater than 500,000 W/cm², or greater than 400,000 W/cm², or greater than 300,000 W/cm², or greater than 200,000 W/cm², or greater than 100,000 W/cm², or greater than 50,000 W/cm², or between about 50,000 W/cm² and about 100,000 W/cm², or between about 50,000 W/cm² and about 250,000 W/cm², or between about 250,000 W/cm² and about 500,000 W/cm², or between about 100,000 W/cm² and about 400,000 W/cm², or between about 350,000 W/cm² and about 500,000 W/cm². In one embodiment, the intensity or field strength of the UV radiation emitted by the laser is about 500,000 W/cm², or about 400,000 W/cm², or about 300,000 W/cm², or about 200,000 W/cm², or about 100,000 W/cm², or about 50,000 W/cm². In such embodiments, the intensity or field strength of the UV radiation incident on the object or a surface thereof may be greater than 500,000 W/cm², or greater than 400,000 W/cm², or greater than 300,000 W/cm², or greater than 200,000 W/cm², or greater than 100,000 W/cm², or greater than 50,000 W/cm², or between about 50,000 W/cm² and about 100,000 W/cm², or between about 50,000 W/cm² and about 250,000 W/cm², or between about 250,000 W/cm² and about 500,000 W/cm², or between about 100,000 W/cm² and about 400,000 W/cm², or between about 350,000 W/cm² and about 500,000 W/cm², or about 500,000 W/cm², or about 400,000 W/cm², or about 300,000 W/cm², or about 200,000 W/cm², or about 100,000 W/cm², or about 50,000 W/cm².

In other embodiments, the intensity or field strength of the UV radiation incident on the object or a surface thereof is a fraction of the intensity or field strength of the UV radiation emitted by the laser. In such embodiments, the intensity or field strength of the UV radiation incident on an object or a surface thereof may be lowered relative to the intensity or field strength of the UV radiation emitted by the laser by dividing the beam into two or more pulses. Division of the beam may be achieved through use of a beam splitter and one or more turning mirror(s). Suitable beam splitters will be known to those of skill in the art. By way of non-limiting example, a beam splitter that divides the original beam into two beams may be used, for example, a beam splitter that divides the beam into two equal energy beams are readily available. Further, a beam splitter that divides the beam into two energy beams of other transmission and reflection ratios are readily available. One Commercial source of a beam splitter is Edmund Optics. In such embodiments, the intensity or field strength of the UV radiation incident on the object or a surface thereof may be greater than 1,000 W/cm², or greater than 2,500 W/cm², or greater than 5,000 W/cm², or greater than 7,500 W/cm², or greater than 10,000 W/cm², or greater than 15,000 W/cm², or greater than 25,000 W/cm², or less than 1,000 W/cm², or less than 2,500 W/cm², or less than 5,000 W/cm², or less than 7,500 W/cm², or less than 10,000 W/cm², or less than 15,000 W/cm², or less than 25,000 W/cm², or between about 1,000 W/cm² and about 7,500 W/cm², or between about 5,000 W/cm² and about 10,000 W/cm², or between about 7,500 W/cm² and about 20,000 W/cm², or between about 10,000 W/cm² and about 25,500 W/cm², or between about 25,000 W/cm² and about 50,000 W/cm², or about 1,000 W/cm², or about 2,500 W/cm², or about 5,000 W/cm², or about 7,500 W/cm², or about 10,000 W/cm², or about 15,000 W/cm², or about 25,000 W/cm².

Any suitable laser pulse duration may be used. In one embodiment, the laser pulse duration is from about 5 to about 35 ns, or from about 5 to about 10 ns, or from about 10 to about 20 ns, or from about 15 to about 25 ns, or from about 20 to about 35 ns, or from about 25 to 30 ns, or from about 12 to about 35 ns, or about 5 ns, 10 ns, 15 ns, 20 ns, 25 ns, 30 ns, or 35 ns. The laser pulse duration may be short enough such that the object being conveyed through the laser beam at a known speed is irradiated by a full pulse in an effectively “frozen” position.

Any suitable laser pulse repetition rate may be used. Excimer lasers allow adjustment of pulse repetition rate. In some embodiments, the laser pulse repetition rate is shortened or lengthened depending on the speed at which an object is conveyed through the laser beam to ensure that a laser pulse irradiates the object. In some embodiments, the laser pulse repetition rate is shortened or lengthened depending on the speed at which multiple objects are conveyed through the laser beam to ensure that a laser pulse irradiates each object. In one embodiment, the laser pulse repetition rate is from about 10 Hz to about 600 Hz, or from about 10 Hz to about 100 Hz, or from about 50 Hz to about 250 Hz, or from about 150 Hz to about 400 Hz, or from about 200 Hz to about 400 Hz, or from about 300 Hz to about 600 Hz, or about 20 Hz, 50 Hz, 100 Hz, 200 Hz, 250 Hz, 300 Hz, 350 Hz, 400 Hz, 500 Hz, or 600 Hz. The laser pulse repetition rate need not be constant. In some embodiments, the laser pulse repetition rate is increased as the speed of the object conveying system increases. By way of non-limiting example, the laser pulse repetition rate may be 100 Hz during start-up of the conveying system or at a speed of less than 1 m/s, and may be increased to 300 Hz when the conveying system reaches full speed or a speed of greater than 4 m/s. In some embodiments, the laser pulse repetition rate is decreased as the speed of the object conveying system decreases.

Each object or surface thereof may be subjected to a single pulse of light. The single pulse of light may be delivered by a single laser source. In some embodiments, a single pulse of light is split into two separate beams using a beam splitting element. In such embodiments, a single pulse may be delivered to the surface of an object through two separate light beams. The two separate light beams may be directed to be incident on the surface of an object when the object is in two different places. The two separate light beams may be directed to be incident on the surface of an object at different angles. The two separate light beams may be directed to be incident on the surface of an object at two different angles and when the object is in two different places. In embodiments where the two separate light beams, both being components of the same laser source UV light beam, are directed to be incident on the surface of an object when the object is in two different places, a delay between a first light beam and a second light beam may be any suitable delay. In some embodiments, the delay may be between about 5 to about 25 ns, or from about 5 to about 10 ns, or from about 10 to about 20 ns, or from about 15 to about 25 ns, or about 5 ns, 10 ns, 15 ns, 20 ns, or 25 ns. In other embodiments, the delay may be between about 1 to about 100 ms, or from about 1 to about 10 ms, or from about 10 to about 25 ms, or from about 25 to about 50 ms, or from about 50 to about 75 ms, or from about 60 to about 85 ms, or from about 75 to about 100 ms, or about 1 ms, 2.5 ms, 5 ms, 10 ms, 25 ms, 50 ms, 75 ms, or 100 ms. In other embodiments, longer delays may be used. Delays may be adjusted using any suitable means, including a delay line, in some embodiments comprising a set of mirrors that run a beam over a known distance before it is entered back into the beam delivery system.

In some embodiments, the object is subjected to a single pulse of light that is partially retroreflected such that two surfaces of the object are irradiated by the single pulse. In such embodiments, a single pulse of light emitted by the UV light source may be expanded such that it illuminates an area approximately twice the size of a first surface of an object. Half the expanded light beam may be incident on the first surface of the object, with the remaining half of the expanded light beam being directed onto a retroreflective mirror positioned underneath the object. Alternatively, half the expanded light beam may be incident on the first surface of the object, with the remaining half of the expanded light beam being directed onto a retroreflective mirror positioned behind or to the rear of the object. By angling the retroreflective mirror towards a second surface of the object, in some embodiments, wherein the second surface opposes the first surface, two surfaces of the same object may be simultaneously irradiated by a single beam pulse. In other embodiments, other fractions of the beam may be retroreflected. By way of non-limiting example, 60% of the beam from the light source may be incident on a first surface of an object, and 40% of the beam may be incident on a retroreflective mirror and be directed to a second surface of the object, however, other fractions may be used such as 70%/30%, 80%/20%, etc. Suitable retroreflective mirrors will be known to those of skill in the art and are commercially available. In some embodiments, the portion of the expanded light beam incident upon the retroreflective mirror is directly incident upon the retroreflective mirror. In some embodiments, this is achieved by expanding the beam past the conveyer system. In other embodiments, the portion of the expanded light beam incident upon the retroreflective mirror is indirectly incident upon the retroreflective mirror. In some embodiments, this is achieved by passing the beam past the object but through an illuminating stage in the conveyer system prior to reaching the retroreflective mirror. In such cases, the illuminating stage may comprise UV transparent material, such as polypropylene or Teflon.

In some embodiments, an unstable resonator is used in conjunction with an excimer laser and retroreflective mirror. An unstable resonator may advantageously reduce divergence of the laser beam when retroreflected onto the second face of the banknote, and thereby reduce the proportion of the beam of UV light lost to the surroundings. Accordingly, use of an unstable resonator may allow illumination of the rear side of an object to be performed by means of a retroreflective mirror without the need to focus the reflected light and/or suffering from a limited uniform illumination area. In some embodiments, an unstable resonator may advantageously reduce divergence of the laser beam by up to a factor of 10, or by at least a factor of 10, or by about a factor of 10, over a standard configuration. In these embodiments, use of an unstable resonator in conjunction with an excimer laser may result in less than 10% of the retroreflected laser beam being lost to the surroundings, or less than 9%, 8%, 7%, 6%, 5%, 4%, 3%, 2%, 1%, or 0.5% of the retroreflected laser beam being lost to the surroundings. In this context, the term “surroundings” refers to an area outside the second face of the banknote. Use of an unstable resonator may advantageously dispense with the need for additional optics to ensure suitable beam homogeneity. Suitable unstable resonators will be known to those of skill in the art. Suitable unstable resonators will be known to those of skill in the art and are commercially available. Unstable resonator optics may be fitted to any excimer laser.

In other embodiments, each object is subjected to two pulses of light. In yet a further embodiment, each object is subjected to more than two pulses of light. In embodiments where an object receives multiple pulses of light, the pulses may have the same wavelength, intensity and pulse duration. Alternatively, in embodiments where an object receives multiple pulses of light, the pulses may have a different wavelength, or may have a different intensity, or may have different pulse duration. In yet other embodiments where an object receives multiple pulses of light, the pulses may have a different wavelength and different intensity and different pulse duration. In one embodiment, an object may receive two pulses of light, a first pulse and a second pulse, wherein the first pulse comprises light having a first wavelength, and the second pulse comprises light having a second wavelength. In one embodiment, an object may receive two pulses of light, a first pulse and a second pulse, wherein the first pulse comprises light having a wavelength of 193 nm, and the second pulse comprises light having a wavelength of 248 nm.

In embodiments where an object receives multiple pulses of light, each of the two or more pulses may be delivered by a single light source. In one embodiment, the single light source is a single laser. In such embodiments, a flipping mirror may be used to direct alternate pulses from the single laser along two separate beam paths to the surface of an object. In other embodiments, a flipping mirror is used to direct successive beam pulses to a single object in two different positions. In some embodiments, the flipping mirror is a single axis galvanometer mirror. In such embodiments, the alternate beam paths or successive pulses are both directed along a common plane, being in some embodiments the plane of object movement. In another embodiment, the flipping mirror is a 2-dimensional galvanometer mirror or mirror set. In such embodiments, the alternate beam paths or successive pulses need not be directed along the plane of object movement. In some embodiments, a flipping mirror is used to direct alternate beam paths or successive pulses to two different objects. Suitable flipping mirrors will be known to those of skill in the art.

In other embodiments, each of the two or more pulses is delivered by two or more different light sources. In one embodiment, the two or more different light sources are two or more separate lasers. In one embodiment, the two or more different light sources are two or more excimer lasers. In such embodiments, a beam from a first laser may be used to irradiate an object at a first location, and a beam from a second laser may be used to irradiate the same object at a second location spaced apart from the first location.

Banknote sorting machines generally singularise notes at speeds of less than 50 Hz, and many singularise notes at a speed of less than 20 Hz. Therefore, in some embodiments, a single laser provides a single pulse, or in other embodiments two or more pulses, to a surface of a banknote while the sorting takes place. In such embodiments, an excimer laser having a pulse repetition rate of about 300 Hz may be advantageous.

As described above, in some embodiments, an unstable resonator is used in conjunction with an excimer laser and a retroreflective mirror. However, in other embodiments, an unstable resonator is used in conjunction with an excimer laser without a retroreflective mirror. In some embodiments, use of an unstable resonator with an excimer laser may require removal of the laser focusing lens. In some embodiments, use of an unstable resonator in conjunction with an excimer laser may advantageously increase beam uniformity (relative to the beam of an excimer laser without an unstable resonator) and thereby eliminate the need for auxiliary beam homogenisation techniques. In some embodiments, use of an unstable resonator removes the need for the excimer laser beam to be focused to a predetermined focal spot. Use of an unstable resonator with an excimer laser may remove positional sensitivity of objects being irradiated. In other words, in embodiments where an unstable resonator is used, the objects irradiated by the unstable resonator beam may be less sensitive to the distance between the object (or a surface thereof) and the excimer laser, which may give greater flexibility in installation and operation of the apparatus and/or methods described herein. Such embodiments may also advantageously enable objects with three-dimensional shape, such as parcels, to be sanitised with a predictable UV light dose, as such objects may not have a uniform size and therefore may not maintain a constant distance between the object (or a surface thereof) and the laser. Use of an unstable resonator may advantageously mean the delivered energy remains constant over the full interaction area in the Z axis, enabling uniform illumination of the surfaces of three-dimensional objects without the need to compensate for object height differences.

In some embodiments, the light pulse is configured to provide uniform illumination, or substantially uniform illumination, to a surface of an object. As used herein, the term “uniform” in relation to illumination of a surface refers to the incident energy on a surface in mJ/cm² being equal within +10%, within +5%, or within +4%, or within +3%, or within +2%, or within ±1%, or within ±0.5%, or within +0.1% at any two given points on a surface. Uniformity of the laser beam may be adjusted from raw laser uniformities of about +10% using any suitable devices. In some embodiments, values of closer to ±3% may be achieved using an unstable resonator or inserted wedge, or values of <1% may be achieved using a crossed cylinder homogeniser.

The light beam may be configured to irradiate a defined surface area by use of a beam expansion lens. In some embodiments, multiple lenses may be used. The beam expansion lens or lenses may be used to increase laser beam uniformity by increasing the focal spot size of the beam and therefore allowing any non-uniform beam edges to irradiate a region beyond the surface of an object. The light pulse may be configured to irradiate a defined surface area by use of a beam expanding telescope. The beam expanding telescope may be used to increase laser beam uniformity by increasing the focal spot size of the beam and therefore allowing any non-uniform beam edges to irradiate a region beyond the surface of an object. Suitable beam expansion lenses or beam expansion telescopes will be known to those of skill in the art. In some embodiments, the imaging lens of the laser is removed, thereby lowering the incident energy of the laser beam. In some embodiments, the imaging lens of the excimer laser is removed. In some embodiments, the UV light irradiates an area corresponding to the size and shape of the surface of the object. In such embodiments, the UV light may irradiate the entirety of a surface of an object in a single pulse. In some embodiments, the UV light irradiates an area corresponding to the size and shape of the surface of the object. In some embodiments, the UV light irradiates an area larger than the size and shape of the surface of the object. In such embodiments, a portion of the beam may not be incident on a surface of an object, instead irradiating in some embodiments the conveyer system or illumination stage. In some embodiments, a portion of the beam may be retroreflected onto a second surface of the object. In some embodiments, the UV light irradiates an area smaller than the size and shape of the surface of the object.

The surface of an object may be exposed to the beam of UV light for any suitable exposure time. In some embodiments, the surface of an object may be exposed to the beam of UV light for an exposure time of one pulse duration. In other embodiments, the surface of an object may be exposed to the beam of UV light for an exposure time of two pulse durations. In yet other embodiments, the surface of an object may be exposed to the beam of UV light for an exposure time of a fraction of a single pulse duration. By way of non-limiting example, the fraction may be 90%, 80%, 70%, 60% or 50%. In some embodiments, the object is on an illumination stage when it is exposed to the beam of UV light. In some embodiments, the object is passing over an illumination stage when it is exposed to the beam of UV light. In some embodiments, the beam of UV light is directed towards an illumination stage.

The apparatus herein comprises a system for conveying the object through the beam which, in some embodiments, is a banknote singularisation apparatus. The methods described herein comprise a step of conveying the object through UV light. In such methods, the system for conveying is not particularly limited, but may be as described herein. In such an apparatus, the banknotes are generally automatically fed into a sorting machine in stacks of about 1000 notes. The sorting machine then feeds off banknotes individually and sends them along a conveyor belt in single file. After sorting and checking, the banknotes are restacked. Banknote singularisation systems comprise an area where banknotes run in single file. This area is often configured as a vertical conveyer, but in some cases may be horizontal. In some embodiments, the apparatus and/or methods herein may be used with a vertical singularisation system, wherein a UV-transparent material is used to convey the banknotes. Although the UV transparent material is not limited, suitable materials may include polypropylene, silica, calcium or magnesium fluorides, or Teflon. In other embodiments, the apparatus and/or methods herein may be used with a horizontal singularisation system. In such embodiments, a single surface may be irradiated, being a surface not in contact with the singularisation system. In such embodiments, two opposing surfaces may be irradiated, wherein the surface in contact with the singularisation system is irradiated through a UV transparent material at an illumination stage. In some embodiments, the apparatus described herein is stationed such that the laser beam is directed to the area in banknote singularisation systems where banknotes run in single file. In such banknote singularisation systems banknotes run in single file may reach speeds of about 10 meters per second. Advantageously, such apparatus and/or methods may be retrofittable to, or suitable for use with, existing banknote singularisation systems without the need for any, or any substantial, modifications.

The beam of UV light may be incident on a surface of an object at any suitable angle. In some embodiments, the beam to UV light is incident on a surface of an object at an angle perpendicular to the surface. In other embodiments, the beam of UV light is incident on a surface of an object at an angle approximately perpendicular to the surface. By way of non-limiting example, an approximately perpendicular angle may be an angle of between about 80° and 90°. The beam of UV light may alternatively be incident on a surface of an object at any other angle, for example, at an angle of between about 5° and about 30°, or at an angle of between about 25° and about 60°, or at an angle of between about 30° and about 45°, or at an angle of between about 45° and about 80°, or at an angle of between about 75° and about 90°, or at an angle of 5°, 10°, 20°, 30°, 40°, 50°, 60°, 70°, 80°, or 90°, or at any angle supplementary to these angles. The beam of UV light may be directed towards a surface of an object at any suitable angle using a turning mirror. In some embodiments, two or more turning mirrors may be used to redirect or angle the beam of UV light. Suitable turning mirrors will be known to those of skill in the art.

In some embodiments, the beam of UV light is not polarised. In other embodiments, the beam of UV light is polarised. In some embodiments, the UV light is polarised using a polarising element. The polarising element may be used to filter the UV light from a laser such that only the S component of the UV beam reaches the surface of an object. In other embodiments, the polarising element may be used to filter the UV light from a laser such that only the P component of the UV beam reaches the surface of an object. In yet further embodiments, a polarising element may be used to filter the UV light from a laser such that the S component and the P component of the UV beam reach the surface of an object separately. Suitable polarising elements will be known to those of skill in the art.

Other UV light sources are contemplated herein. Artificial radiation UV light sources contemplated herein include UV lamps and the like. However, laser sources may be advantageous due to their relatively higher intensity and therefore shorter exposure times required to deliver an equivalent energy to a surface. In one embodiment, radiation from a UV lamp may be in the form of continuous wave radiation having a wavelength of 254 nm. Intensities of the order of 100 μW/cm² may be achieved with a UV lamp. In such embodiments, where a UV lamp is used, exposure times of greater than 10 ms, or greater than 100 ms, or greater than 1 s, or greater than 10 s, or greater than 20 s, or greater than 30 s, or greater than 40 s, or greater than 50 s, or greater than 60 s, or greater than 70 s, or greater than 80 s, or greater than 90 s may be required, or exposure times of from about 10 s to about 100 s, or of from about 20 s to about 50 s, or of from about 30 s to about 70 s, or of from about 50 s to about 75 s, or of from about 80 s to about 100 s may be required. UV lamps may deliver energies of from about 0.1 mJ/cm² to about 10 mJ/cm² to surfaces. Higher doses are possible given sufficient exposure times.

In some embodiments, a trigger system configured to detect the presence of an object is used to command the UV light source to emit a beam of UV light to a surface of the object. The trigger system may comprise a camera or motion-sensitive device that detects the presence of an object on a predetermined illumination stage and relays a signal to the UV light source to emit a beam of UV light. Suitable trigger systems will be known to those of skill in the art.

In some embodiments, a scanning mirror may be used to direct a beam along a single axial path so that the beam can be placed at any position within the field of view of the optical system. In some embodiments, a scanning mirrors can be configured to be a flipping mirror for repetitive applications. In related embodiments, a flipping mirror may be configured to move to one of two or three or more locations. The apparatus and/or methods described herein may use a single scanning mirror, or may use two or more scanning mirrors. Suitable scanning mirrors will be known to those of skill in the art.

In some embodiments, the beam of the UV light is enclosed for safety. In some embodiments, where the UV light source is a laser, the laser and its beam path are enclosed by a shield. The shield may comprise any suitable material that is opaque to (or is able to absorb) UV light. In some embodiments, the shield material is or comprises metal. In some embodiments, the shield is or comprises a box.

Sanitising—Microorganisms and/or Viruses

The methods and/or apparatus herein are suitable for sanitising one or more surfaces of an object. In some embodiments, the methods and apparatus herein are suitable for sanitising an object by reducing the number of viable microorganisms and/or viruses on a surface thereof. A viable microorganism may refer to the microorganism having an intact cell membrane, metabolic activity and/or the ability to reproduce. A viable virus may refer to the virus being capable of reproducing inside a host cell, in some embodiments, a human host cell. In some embodiments, the methods and apparatus herein reduce the number of viable microorganisms and/or viruses on a surface thereof by at least 50%, or by at least 60%, or by at least 70%, or by at least 80%, or by at least 90%, or by at least 95%, or by at least 99%, or by at least 99.9%, or by at least 99.99%, or by at least 99.999%, or by at least 99.9999%, or by at least 99.99999%. In a related embodiment, the methods and apparatus herein reduce the number of viable viruses on a surface thereof by at least 50%, or by at least 60%, or by at least 70%, or by at least 80%, or by at least 90%, or by at least 95%, or by at least 99%, or by at least 99.9%, or by at least 99.99%, or by at least 99.999%, or by at least 99.9999%, or by at least 99.99999%. In a further related embodiment, the methods and/or apparatus herein reduce the number of viable viruses on a surface thereof by at least 95% or by at least 99%, or by at least 99.9%, or by at least 99.99%, or by at least 99.999%, or by at least 99.9999%, or by at least 99.99999%.

In a related embodiment, the reduction in the number of viable microorganisms and/or viruses on a surface is determined by comparing the number of viable microorganisms and/or viruses before and after sanitising an object. In a related embodiment, the number of viable microorganisms and/or viruses is determined before sanitising and at a sufficient time after sanitising the object such that a reduction can be detected. In a related embodiment, the number of viable microorganisms and/or viruses is determined before sanitising and less than about 30 minutes after sanitising the object, before sanitising and less than about 60 minutes after sanitising the object, or before sanitising and less than about 60 minutes after sanitising the object. In some embodiments, the number of viable microorganisms and/or viruses is determined before sanitising and between about 15 minutes and about 90 minutes after sanitising the object, before sanitising and between about 30 minutes and about 90 minutes after sanitising the object or before sanitising and between about 30 minutes and about 60 minutes after sanitising the object.

In some embodiments, the methods and/or apparatus herein are suitable for sanitising an object by reducing the detectable microorganisms and/or viruses on a surface thereof. In some embodiments, the methods and apparatus herein reduce the detectable microorganisms and/or viruses on a surface thereof by at least 50%, or by at least 60%, or by at least 70%, or by at least 80%, or by at least 90%, or by at least 95%, or by at least 99%, or by at least 99.9%, or by at least 99.99%, or by at least 99.999%, or by at least 99.9999%, or by at least 99.99999%. In a related embodiment, the methods and apparatus herein reduce the detectable viruses on a surface thereof by at least 50%, or by at least 60%, or by at least 70%, or by at least 80%, or by at least 90%, or by at least 95%, or by at least 99%, or by at least 99.9%, or by at least 99.99%, or by at least 99.999%, or by at least 99.9999%, or by at least 99.99999%. In a further related embodiment, the methods and apparatus herein reduce the detectable viruses on a surface thereof by at least 95% or by at least 99%, or by at least 99.9%, or by at least 99.99%, or by at least 99.999%, or by at least 99.9999%, or by at least 99.99999%.

In a related embodiment, the reduction in detectable microorganisms and/or viruses on a surface is determined by comparing the detectable microorganisms and/or viruses before and after sanitising an object. In a related embodiment, the presence or absence of the microorganism and/or virus is detected before sanitising and at a sufficient time after sanitising the object such that a reduction can be detected. In a related embodiment, the presence or absence of the microorganism and/or virus is detected before sanitising and less than about 30 minutes after sanitising the object, before sanitising and less than about 60 minutes after sanitising the object, or before sanitising and less than about 60 minutes after sanitising the object. In some embodiments, the presence or absence of the microorganism and/or virus is detected before sanitising and between about 15 minutes and about 90 minutes after sanitising the object, before sanitising and between about 30 minutes and about 90 minutes after sanitising the object or before sanitising and between about 30 minutes and about 60 minutes after sanitising the object.

In some embodiments, the methods and/or apparatus herein sanitise the surface of an object by inactivating microorganisms and/or viruses on the surface. The term “inactivate” and related terms such as “inactivating” or “inactivated” in the context of a particular species of microorganism or type of virus on a surface refers to at least a 1 log 10 reduction in the number of viable microorganisms or viruses in that population on the surface after irradiation with UV light as described herein compared to prior to irradiation. In some embodiments, inactivation refers to at least a 2 log 10 reduction, or at least a 3 log 10 reduction, or at least a 4 log 10 reduction, or at least a 5 log 10 reduction, or at least a 6 log 10 reduction in the number of viable microorganisms or viruses in a given population on a surface after irradiation with UV light as described herein compared to prior to irradiation.

Methods of quantifying microorganism or virus on a surface are not particularly limited and include any method known in the art. In one embodiment real time PCR (also referred to as quantitative PCR (qPCR)) is used. Use of real time PCR to identify and quantify a microorganism and/or a virus may be as described Kralik and Ricchi (2017) Front Microbiol., 8, 108, the contents of which is incorporated herein in full by cross-reference. In one embodiment, for the detection and/or quantification of an RNA virus, reverse-transcriptase polymerase chain reaction (RT-PCR) is used. Use of RT-PCR for detection and/or quantification of an RNA virus may be as described/and or adapted from Corman et al. (2012) Euro Surveill, 17(49), 20334, the contents of which is incorporated herein in full by cross-reference. Use of RT-PCR for detection and/or quantification of SARS-CoV-2 may be as described in Corman et al. (2020) Euro Surveill 25(3), 2000045, the contents of which is incorporated herein in full by cross-reference. In another embodiment, the spot-titre method is used to count plaque forming units (PFUs; e.g. viruses, bacteriophages) or colony forming units (CFUs; bacteria). Spot-titre PFU/CFU assays are variants of the gold standard PFU and CFU assays, which enumerate infectious virus and live bacteria respectively. Utilising a double overlay agar when conducting PFU assays is the gold standard for enumerating bacteriophage. The spot-titre variants of PFU and CFU assays have been shown to maintain very high accuracy compared to the traditional method whilst permitting substantial reductions in reagent usage and processing time. Serial dilutions of microorganism or virus, such as the amount remaining on a surface after UV irradiation, may be made in media and spotted onto an appropriate media plate, which is then cultured for 24 hours at 37° C. and the appropriate dilution determined and counted for each sample to calculate concentration. In some embodiments, the appropriate media plate is LB agar or double agar overlay of TNT agar with host. Other methods of identifying, quantifying and/or assessing the viability of microorganisms and viruses may include bacterial and viral culturing processes, viability PCR, live/dead staining, molecular viability testing (MVT), ELISA, ATP assays (see Weigel et al. (2017) Appl. Environ. Microbiol., 83, 1; Cangelosi and Meschke (2014) Appl. Environ. Microbiol., 80, 5884; Verma et al. (2013) In: Arora D., Das S., Sukumar M. (eds) Analyzing Microbes. Springer Protocols Handbooks. Springer, Berlin, Heidelberg, each of which is incorporated in its entirety by cross-reference).

Any suitable microorganism or virus may be irradiated by UV light using the apparatus and/or methods described herein. In some embodiments, the surface of the object herein may comprise one or more microorganisms. In some embodiments, the surface of the objects herein may comprise one or more viruses. In some embodiments, the surface of the objects herein may comprise one or more microorganisms and one or more viruses. In some embodiments, the microorganism is pathogenic to humans. The microorganisms inactivated by the methods and apparatus described herein are not particularly limited. In some embodiments, the microorganism includes any one or more of the following: Escherichia coli, Bacillus spp., Staphylococcus spp. (including Staphylococcus aureus), coagulase-negative staphylococci, Pseudomonas spp. (including Pseudomonas aeruginosa), Salmonella spp., Enterococcus spp., Streptococcus spp., Klebsiella spp., Proteus mirabilis, Acinetobacter spp., Aerobacter spp., Yersinia spp., Enterobacter spp., Shigella spp. (including Shigella flexneri, Shigella sonnei), Vibrio spp., Aspergillus spp., Candida spp., Fusarium spp., Rhizopus spp., Penicillium spp., Ascaris lumbricoides, Enterobius vermicularis, Trichuris trichiura, Taenia spp., Campylobacter spp., Clostridium perfringens, Listeria monocytogenes, Mycobacterium tuberculosis. Other microorganisms suitably inactivated by the methods and apparatus described herein will be apparent to one of skill in the art. The viruses inactivated by the methods and apparatus described herein are not particularly limited. In some embodiments, the virus is pathogenic to humans. In some embodiments, the virus includes a virus of any one or more of the following types: Adenoviridae, Picornaviridae (including enterovirus, coxsackie virus, rhinovirus, poliovirus), Herpesviridae (including herpes simplex virus, varicella zoster virus, cytomegalovirus), Hepadnaviridae, Caliciviridae (including norovirus), Coronaviridae (including SARS-CoV-2, MERS-Cov, SARS-CoV), Flaviviridae (including Dengue virus), Filoviridae (including Ebola virus), Reoviridae (including rotavirus), Rhadboviridae, Retroviridae (including HIV), Orthomyxoviridae (including Influenza, H1N1 influenza, Influenza B), Paramyxoviridae (including measles virus, mumps virus), Papovaviridae (including HPV), Polyomavirus, Poxviridae, Rhabdoviridae, Togaviridae. Other viruses suitably inactivated by the methods and apparatus described herein will be apparent to one of skill in the art. In some embodiments, the virus is an RNA virus. In some embodiments, the virus is a Coronaviridae. In some embodiments, the virus is SARS-CoV-2.

An initial level of contamination by microorganisms and/or viruses on a surface of an object is not particularly limited in the apparatus and/or methods described herein. However, in some embodiments, an initial level of contamination of microorganisms and/or viruses (that is, a level of contamination on the object prior to irradiation using the apparatus and/or methods herein) may be up to 5×10⁵ PFU/mm², or up to 1×10⁵ PFU/mm², or up to 5×10⁴ PFU/mm², or up to 1×10⁴ PFU/mm², or up to 5×10³ PFU/mm², or up to 1×10³ PFU/mm², or up to 5×10² PFU/mm², or up to 1×10² PFU/mm², or up to 5×10¹ PFU/mm², or up to 1×10¹ PFU/mm², or may be at least 5×10⁵ PFU/mm², or at least 1×10⁵ PFU/mm², or at least 5×10⁴ PFU/mm², or at least 1×10⁴ PFU/mm², or at least 5×10³ PFU/mm², or at least 1×10³ PFU/mm², or at least 5×10² PFU/mm², or at least 1×10² PFU/mm², or at least 5×10¹ PFU/mm², or at least 1×10¹ PFU/mm², or may be between 5×10⁵ PFU/mm² and 1×10¹ PFU/mm², or between 5×10⁵ PFU/mm² and 5×10³ PFU/mm², or between 5×10⁴ PFU/mm² and 5×10² PFU/mm², or may be about 5×10⁵ PFU/mm², 1×10⁵ PFU/mm², 5×10⁴ PFU/mm², 1×10⁴ PFU/mm², 5×10³ PFU/mm², 1×10³ PFU/mm², 5×10² PFU/mm², 1×10² PFU/mm², 5×10¹ PFU/mm², or 1×10¹ PFU/mm², or may be up to 5×10⁶ CFU/cm², or up to 1×10⁶ CFU/cm², 5×10⁵ CFU/cm², or up to 1×10⁵ CFU/cm², or up to 5×10⁴ CFU/cm², or up to 1×10⁴ CFU/cm², or up to 5×10³ CFU/cm², or up to 1×10³ CFU/cm², or up to 5×10² CFU/cm², or up to 1×10² CFU/cm², or up to 5×10¹ CFU/cm², or up to 1×10¹ CFU/cm², or may be at least 5×10⁵ CFU/cm², or at least 1×10⁵ CFU/cm², or at least 5×10⁴ CFU/cm², or at least 1×10⁴ CFU/cm², or at least 5×10³ CFU/cm², or at least 1×10³ CFU/cm², or at least 5×10² CFU/cm², or at least 1×10² CFU/cm², or at least 5×10¹ CFU/cm², or at least 1×10¹ CFU/cm², or may be between 5×10⁵ CFU/cm² and 1×10¹ CFU/cm², or between 5×10⁵ CFU/cm² and 5×10³ CFU/cm², or between 5×10⁴ CFU/cm² and 5×10² CFU/cm², or may be about 5×10⁵ CFU/cm², 1×10⁵ CFU/cm², 5×10⁴ CFU/cm², 1×10⁴ CFU/cm², 5×10³ CFU/cm², 1×10³ CFU/cm², 5×10² CFU/cm², 1×10² CFU/cm² 5×10¹ CFU/cm², or 1×10¹ CFU/cm².

The energy delivered by the light source may be any suitable delivered energy capable of reducing the number of viable microorganisms and/or viruses on a surface of an object. In one embodiment, the energy incident on a surface of an object is at least 0.1 mJ/cm², 0.5 mJ/cm², or at least 1.0 mJ/cm², or at least 1.5 mJ/cm², or at least 2.0 mJ/cm², or at least 2.5 mJ/cm², or at least 3.5 mJ/cm², or at least 4.5 mJ/cm², or at least 5.5 mJ/cm², or at least 6.5 mJ/cm², or at least 7.5 mJ/cm², or at least 8.5 mJ/cm², or at least 9.5 mJ/cm², or at least 10 mJ/cm², or at least 20 mJ/cm², or at least 30 mJ/cm², or at least 50 mJ/cm², or at least 100 mJ/cm², or at least 250 mJ/cm², or at least 500 mJ/cm², or at least 1000 mJ/cm², or is between about 0.1 mJ/cm² and about 0.5 mJ/cm², or is between about 0.5 mJ/cm² and about 2.5 mJ/cm², or is between about 2.5 mJ/cm² and about 7.5 mJ/cm², or is between about 5 mJ/cm² and about 10 mJ/cm², or is between about 5 mJ/cm² and about 20 mJ/cm², or is between about 10 mJ/cm² and about 50 mJ/cm², or is between about 50 mJ/cm² and about 100 mJ/cm², or is between about 100 mJ/cm² and about 500 mJ/cm², or is between about 500 mJ/cm² and about 1000 mJ/cm², or is about 0.1 mJ/cm², 0.5 mJ/cm², 1.0 mJ/cm², 1.5 mJ/cm², 2.0 mJ/cm², 2.5 mJ/cm², 3.5 mJ/cm², 4.5 mJ/cm², 5.5 mJ/cm², 6.5 mJ/cm², 7.5 mJ/cm², 8.5 mJ/cm², 9.5 mJ/cm², 10 mJ/cm², 20 mJ/cm², 30 mJ/cm², 50 mJ/cm², 100 mJ/cm², 250 mJ/cm², 500 mJ/cm², or 1000 mJ/cm². This energy may be delivered to the surface of the object by a single pulse of UV light of variable duration, but in some embodiments, may be delivered by two or more pulses of UV light of variable duration to the same surface. Other parameters relating to delivery of UV light to the surface are discussed in more detail below.

The energy delivered by the light source may be any suitable delivered energy capable of reducing the microorganisms and/or viruses on a surface of an object. In one embodiment, the energy incident on a surface of an object is at least 0.1 mJ/cm², or at least 0.5 mJ/cm², or at least 1.0 mJ/cm², or at least 1.5 mJ/cm², or at least 2.0 mJ/cm², or at least 2.5 mJ/cm², or at least 3.5 mJ/cm², or at least 4.5 mJ/cm², or at least 5.5 mJ/cm², or at least 6.5 mJ/cm², or at least 7.5 mJ/cm², or at least 8.5 mJ/cm², or at least 9.5 mJ/cm², or at least 10 mJ/cm², or at least 20 mJ/cm², or at least 30 mJ/cm², or at least 50 mJ/cm², or at least 100 mJ/cm², or at least 250 mJ/cm², or at least 500 mJ/cm², or at least 1000 mJ/cm², or is between about 0.1 mJ/cm² and about 2.5 mJ/cm², or is between about 2.5 mJ/cm² and about 7.5 mJ/cm², or is between about 5 mJ/cm² and about 10 mJ/cm², or is between about 5 mJ/cm² and about 20 mJ/cm², or is between about 10 mJ/cm² and about 50 mJ/cm², or is between about 50 mJ/cm² and about 100 mJ/cm², or is between about 100 mJ/cm² and about 500 mJ/cm², or is between about 500 mJ/cm² and about 1000 mJ/cm², or is about 0.5 mJ/cm², 1.0 mJ/cm², 1.5 mJ/cm², 2.0 mJ/cm², 2.5 mJ/cm², 3.5 mJ/cm², 4.5 mJ/cm², 5.5 mJ/cm², 6.5 mJ/cm², 7.5 mJ/cm², 8.5 mJ/cm², 9.5 mJ/cm², 10 mJ/cm², 20 mJ/cm², 30 mJ/cm², 50 mJ/cm², 100 mJ/cm², 250 mJ/cm², 500 mJ/cm², or 1000 mJ/cm².

In one embodiment, the energy delivered by the light source to the surface of the object and suitable for reducing the microorganisms and/or viruses on the surface is at least 1.0 mJ/cm², at least 1.5 mJ/cm², or at least 2.0 mJ/cm², or at least 2.5 mJ/cm², or at least 3.0 mJ/cm², or at least 3.5 mJ/cm², or at least 4 mJ/cm², or at least 4.5 mJ/cm², or is between about 3.5 mJ/cm² and about 5.0 mJ/cm², or is between about 1.0 mJ/cm² and about 5.0 mJ/cm², or is between about 1.0 mJ/cm² and about 4.0 mJ/cm², or is between about 2.0 mJ/cm² and about 4.5 mJ/cm², or is about 1.0 mJ/cm², 1.5 mJ/cm², 2.0 mJ/cm², 2.5 mJ/cm², 3.0 mJ/cm², 3.5 mJ/cm², 4.0 mJ/cm², or 4.5 mJ/cm² at a wavelength of 248 nm. In another embodiment, the energy delivered by the light source to the surface of the object and suitable for reducing the microorganisms and/or viruses on the surface is at least 1.0 mJ/cm², or at least 1.5 mJ/cm², or at least 2.0 mJ/cm², or at least 2.5 mJ/cm², or at least 3.0 mJ/cm², or is between about 2.0 mJ/cm² and about 3.0 mJ/cm², or is between about 1.0 mJ/cm² and about 3.5 mJ/cm², or is between about 1.0 mJ/cm² and about 4.0 mJ/cm², or is about 1.0 mJ/cm², 1.5 mJ/cm², 2.0 mJ/cm², 2.5 mJ/cm², 3.0 mJ/cm², or 3.5 mJ/cm² at a wavelength of 193 nm.

As described herein, reduction of microorganism or virus on a surface is achieved by exposing the microorganisms and/or viruses to UV radiation that can be absorbed by molecules in their DNA or RNA and thereby disrupt the DNA or RNA structure. Disruptions in the DNA or RNA structure may then result in damage to the microorganism cell membrane, inability of the microorganism to carry out metabolic processes, and/or inability of the microorganism or virus to reproduce. It will be appreciated that different microorganisms and viruses will require different doses of UV radiation in order to achieve a given reduction in the viable population and that the UV may impact the microorganism or virus viability in different ways depending on the species or type. In some embodiments, the dose response of a particular microorganism or virus is determined prior to using the methods and/or apparatus described herein. In such embodiments, a dose response may be determined by correlating the log reduction in viability of a population of a given microorganism or virus as a function of energy of UV light in mJ/cm² at a given wavelength for a given surface (or type thereof).

In some embodiments, the energy delivered to a surface may be of a quantity sufficient to inactivate all microorganisms and/or viruses present on that surface. In some embodiments, the energy delivered to a surface may be of a quantity sufficient to inactivate all pathogenic microorganisms and/or viruses present on that surface. In this context, “pathogenic” refers to the microorganism or virus being capable of causing disease in a human. In some embodiments, the energy delivered by the light source is of a quantity sufficient to inactivate SARS-Cov-2 virus present on a given surface.

### Sanitising—Drug Substance

The methods and/or apparatus herein are suitable for sanitising one or more surfaces of an object. In some embodiments, the methods and apparatus herein are suitable for sanitising an object by reducing a concentration of a drug substance on a surface thereof.

In some embodiments, the methods and apparatus herein reduce a concentration of a drug substance on a surface thereof by at least 25%, or at least 50%, or by at least 60%, or by at least 70%, or by at least 80%, or by at least 90%, or by at least 95%, or by at least 99%, or by at least 99.9%, or by at least 99.99%, or by at least 99.999%, or by at least 99.9999%, or by at least 99.99999%. The reduction in a concentration of a drug substance on a surface is determined by comparing the concentration of a drug before and after sanitising an object.

In some embodiments, the methods and/or apparatus herein sanitise the surface of an object by reducing a concentration of a drug substance on the surface by altering the chemical structure of the drug substance. The term “altering” and related terms such as “alter” or “altered” in the context of a structure of a drug substance refers to changing the chemical structure of the drug substance such that it can no longer be characterised as the drug substance, and/or can no longer exert its physiological effects. Methods of quantifying a concentration of a drug substance on a surface are not particularly limited and include any method known in the art. In one embodiment, spectrometric methods are utilised. Spectrometric methods include mass spectrometry, ion mobility spectrometry, and infra-red (IR) spectrometry. In other embodiments, other methods may be utilised. X-ray diffractometry, Raman spectroscopy, UV-Visible spectroscopy, chromatographic techniques including gas and liquid chromatography, colorimetric testing, and immunoassays, may be used. In some embodiments, ELISA-based assays may be used. In yet further embodiments, combinations of two or more methods may be utilised. In one embodiment, gas chromatography-mass spectrometry (GC-MS) may be used to identify and quantify a drug substance. In another embodiment, colorimetric methods utilising non-specific oxidation of the drug substance and coincident reduction of purple potassium permanganate (KMnO₄) to green MnO₄ manganate ions under alkaline conditions may be used. In such embodiments, assessment of the kinetics of the colorimetric reaction and comparison to a standard curve of drug concentrations permits interpolation of the concentration of the drug in an unknown solution from the absorbance at 633 nm. Concentration of drug substance may be quantified spectrophotometrically, such as at 633 nm, at set times after reacting a solution containing a series of known concentrations of drug substance with sodium hydroxide and potassium permanganate and subsequently a standard curve generated. Methods of obtaining calibration curves and standard or reference samples will be known to those of skill in the art. In other embodiments, saliva and/or urine tests for commonly tested illicit drug substances in Australia may be used. In one embodiment, a commercially available saliva test (detection limit of 50 ng/mL amphetamine) or urine (detection limit of 300 ng/mL amphetamine) test kit may be used. Such kits are available from MediNat Australia. In some embodiments, instructions accompanying the commercially available kits may be followed to determine the presence and/or concentration of a drug substance before and after sanitising according to the methods described herein.

Any suitable drug substance may be irradiated by UV light using the apparatus and/or methods described herein. In some embodiments, the surface of the object herein may comprise one or more different drug substances. A drug substance is any substance (other than food that provides nutritional support) that, when inhaled, injected, smoked, consumed, ingested, absorbed via a patch on the skin, absorbed via a mucus membrane, or dissolved under the tongue causes a physiological (and often psychological) change in the body. The drug substance inactivated by the methods and apparatus described herein is not particularly limited. As used herein “drug substance” and related terms such as “drug substances” or “drug” includes a controlled drug substance and/or an illicit substance. “Controlled” in this context may be with reference to a government regulation controlling its use and/or requiring prescription by a medical professional. “Illicit” in this context may be with reference to any national or international law. The skilled person will understand that depending on the location and applicable law, a drug substance may be a controlled drug substance or an illicit drug substance, or may be both a controlled drug substance and an illicit drug substance. In some embodiments, a drug substance may be both a controlled drug substance and an illicit drug substance. In some embodiments, the drug substance is an illicit drug substance.

In some embodiments, the drug substance includes one or more of an opiate compound, an amphetamine compound (including a methamphetamine compound), a cannabis compound (including a cannabinoid compound), a cocaine compound, heroin, ketamine, and/or lysergic acid diethylamide (LSD). In other embodiments, the illicit drug substance includes an opiate compound, an amphetamine compound (including a methamphetamine compound), a cannabis compound (including a cannabinoid compound), a cocaine compound, heroin, ketamine, and/or lysergic acid diethylamide (LSD). In one embodiment, a cocaine compound is cocaine in a crystalline (HCl salt), free base, or “crack” form, or a combination thereof. As used herein, the term “cocaine” should be understood as referring to a “cocaine compound” as described herein. In one embodiment, an amphetamine compound includes amphetamine, paramethoxyamphetamine, and methamphetamines such as 3,4-methylenedioxymethamphetamine, in crystal or powder form, or a combination thereof. In one embodiment, a methamphetamine compound is methamphetamine in crystal or powder form, or MDMA (3,4-methyl-enedioxy-methamphetamine), or a combination thereof. As used herein, the term “methamphetamine” should be understood as referring to a “methamphetamine compound” as described herein. In one embodiment, a cannabis compound is a cannabinoid compound, including delta-9-tetrahydrocannabinol (THC) or cannabidiol (CBD), or a combination thereof. As used herein, the term “cannabis” should be understood as referring to a “cannabis compound” as described herein. Other drug substances suitably inactivated by the methods and apparatus described herein will be apparent to one of skill in the art. In some embodiments, the drug substance inactivated by the methods and apparatus described herein have non-zero absorption of light in the UV part of the spectrum (between 10 and 400 nm). In some embodiments, the drug substance may contain an aromatic and/or an extended π-electron system capable of absorbing UV radiation.

An initial level of contamination by drug substance on a surface of an object is not particularly limited in the apparatus and/or methods described herein. However, in some embodiments, an initial level of contamination by a drug substance (that is, a level of contamination on the object prior to irradiation using the apparatus and/or methods herein) may be up to 250 ng/mm², or up to 200 ng/mm², or up to 100 ng/mm², or up to 50 ng/mm², or up to 25 ng/mm², or up to 10 ng/mm², or up to 1 ng/mm², or up to 250 pg/mm², or up to 200 pg/mm², or up to 100 pg/mm², or up to 50 pg/mm², or up to 25 pg/mm², or up to 10 pg/mm², or up to 1 pg/mm², or up to 0.25 pg/mm², or up to 0.20 pg/mm², or up to 0.1 pg/mm², or up to 0.050 pg/mm², or may be at least 250 ng/mm², or at least 200 ng/mm², or at least 100 ng/mm², or at least 50 ng/mm², or at least 25 ng/mm², or at least 10 ng/mm², or at least 1 ng/mm², or at least 250 pg/mm², or at least 200 pg/mm², or at least 100 pg/mm², or at least 50 pg/mm², or at least 25 pg/mm², or at least 10 pg/mm², or at least 1 pg/mm², or at least 0.25 pg/mm², or at least 0.20 pg/mm², or at least 0.1 pg/mm², or at least 0.050 pg/mm², or may be between 250 ng/mm² and 0.05 pg/mm², or between 100 ng/mm² and 1 pg/mm², or between 50 ng/mm² and 0.05 pg/mm², or may be about 250 ng/mm², 200 ng/mm², 100 ng/mm², 50 ng/mm², 25 ng/mm², 10 ng/mm², 1 ng/mm², 250 pg/mm², 200 pg/mm², 100 pg/mm², 50 pg/mm², 25 pg/mm², 10 pg/mm², 1 pg/mm², 0.25 pg/mm², 0.20 pg/mm², 0.1 pg/mm², or about 0.050 pg/mm². In some embodiments, an initial level of contamination by a drug substance (that is, a level of contamination on the object prior to irradiation using the apparatus and/or methods herein) may be 1 mg/note, or up to 500 μg/note, or up to 400 μg/note, or up to 300 μg/note, or up to 200 μg/note, or up to 100 μg/note, or up to 50 μg/note, or up to 10 μg/note, or up to 1 μg/note, or up to 750 ng/note, or up to 500 ng/note, or up to 250 ng/note, or up to 100 ng/note, or up to 50 ng/note, or up to 25 ng/note, or up to 10 ng/note, or up to 1 ng/note, or may be at least 1 mg/note, or at least 500 μg/note, or at least 400 μg/note, or at least 300 μg/note, or at least 200 μg/note, or at least 100 μg/note, or at least 50 μg/note, or at least 10 μg/note, or at least 1 μg/note, or at least 750 ng/note, or at least 500 ng/note, or at least 250 ng/note, or at least 100 ng/note, or at least 50 ng/note, or at least 25 ng/note, or at least 10 ng/note, or at least 1 ng/note, or between 1 mg/note and 100 μg/note, or between 750 μg/note and 50 μg/note, or between 350 μg/note and 1 μg/note, or between 100 μg/note and 100 ng/note, or between 1 μg/note and 1 ng/note, or between 500 ng/note and 1 ng/note, 1 mg/note, or 500 μg/note, or 400 μg/note, or 300 μg/note, or 200 μg/note, or 100 μg/note, or 50 μg/note, or 10 μg/note, or 1 μg/note, or 750 ng/note, or 500 ng/note, 250 ng/note, or 200 ng/note, or 100 ng/note, or 50 ng/note, or 25 ng/note, or 10 ng/note, or 1 ng/note.

The energy delivered by the light source may be any suitable delivered energy capable of reducing a concentration of a drug substance on a surface of an object. In one embodiment, the energy incident on a surface of an object is at least 0.1 mJ/cm², 0.5 mJ/cm², or at least 1.0 mJ/cm², or at least 1.5 mJ/cm², or at least 2.0 mJ/cm², or at least 2.5 mJ/cm², or at least 3.5 mJ/cm², or at least 4.5 mJ/cm², or at least 5.5 mJ/cm², or at least 6.5 mJ/cm², or at least 7.5 mJ/cm², or at least 8.5 mJ/cm², or at least 9.5 mJ/cm², or at least 10 mJ/cm², or at least 20 mJ/cm², or at least 30 mJ/cm², or at least 50 mJ/cm², or at least 100 mJ/cm², or at least 250 mJ/cm², or at least 500 mJ/cm², or at least 1000 mJ/cm², or is between about 0.1 mJ/cm² and about 0.5 mJ/cm², or is between about 0.5 mJ/cm² and about 2.5 mJ/cm², or is between about 2.5 mJ/cm² and about 7.5 mJ/cm², or is between about 5 mJ/cm² and about 10 mJ/cm², or is between about 5 mJ/cm² and about 20 mJ/cm², or is between about 10 mJ/cm² and about 50 mJ/cm², or is between about 50 mJ/cm² and about 100 mJ/cm², or is between about 100 mJ/cm² and about 500 mJ/cm², or is between about 500 mJ/cm² and about 1000 mJ/cm², or is about 0.1 mJ/cm², 0.5 mJ/cm², 1.0 mJ/cm², 1.5 mJ/cm², 2.0 mJ/cm², 2.5 mJ/cm², 3.5 mJ/cm², 4.5 mJ/cm², 5.5 mJ/cm², 6.5 mJ/cm², 7.5 mJ/cm², 8.5 mJ/cm², 9.5 mJ/cm², 10 mJ/cm², 20 mJ/cm², 30 mJ/cm², 50 mJ/cm², 100 mJ/cm², 250 mJ/cm², 500 mJ/cm², or 1000 mJ/cm². This energy may be delivered to the surface of the object by a single pulse of UV light of variable duration, but in some embodiments, may be delivered by two or more pulses of UV light of variable duration to the same surface.

In one embodiment, the energy delivered by the light source to the surface of the object and suitable for reducing a concentration of drug substance on the surface is at least 1.0 mJ/cm², at least 1.5 mJ/cm², or at least 2.0 mJ/cm², or at least 2.5 mJ/cm², or at least 3.0 mJ/cm², or at least 3.5 mJ/cm², or at least 4 mJ/cm², or at least 4.5 mJ/cm², or is between about 3.5 mJ/cm² and about 5.0 mJ/cm², or is between about 1.0 mJ/cm² and about 5.0 mJ/cm², or is between about 1.0 mJ/cm² and about 4.0 mJ/cm², or is between about 2.0 mJ/cm² and about 4.5 mJ/cm², or is about 1.0 mJ/cm², 1.5 mJ/cm², 2.0 mJ/cm², 2.5 mJ/cm², 3.0 mJ/cm², 3.5 mJ/cm², 4.0 mJ/cm², or 4.5 mJ/cm² at a wavelength of 248 nm. In another embodiment, the energy delivered by the light source to the surface of the object and suitable for reducing a concentration of drug substance on the surface is at least 1.0 mJ/cm², or at least 1.5 mJ/cm², or at least 2.0 mJ/cm², or at least 2.5 mJ/cm², or at least 3.0 mJ/cm², or is between about 2.0 mJ/cm² and about 3.0 mJ/cm², or is between about 1.0 mJ/cm² and about 4.0 mJ/cm², or is between about 1.0 mJ/cm² and about 3.5 mJ/cm², or is about 1.0 mJ/cm², 1.5 mJ/cm², 2.0 mJ/cm², 2.5 mJ/cm², 3.0 mJ/cm², or 3.5 mJ/cm² at a wavelength of 193 nm.

As described herein, reducing a concentration of a drug substance is achieved by exposing the drug substance to UV radiation that can be absorbed by molecules of the drug substance and thereby alter their chemical structure. Alteration of chemical structure may then result in loss of chemical characteristics of the drug substance, such that it is no longer detectable as the drug substance, and/or result in loss of activity of the drug substance, such that it no longer exerts any physiological effects in humans. In some embodiments, a reduction in concentration of drug substance of up to 100%, or up to 90%, or up to 80%, or up to 70% or up to 60%, or up to 50%, or up to 40%, or up to 30%, or up to 20% or up to 10% may be achieved in certain embodiments of the apparatus and/or methods described herein. In some embodiments, a reduction in concentration of drug substance of at least 100%, or at least 90%, or at least 80%, or at least 70% or at least 60%, or at least 50%, or at least 40%, or at least 30%, or at least 20% or at least 10% may be achieved in certain embodiments of the apparatus and/or methods described herein. In some embodiments, a reduction in concentration of drug substance of between 10% and 100%, or from 10% to 50%, or from 15% to 65%, or from 25% to 75%, or from 50% to 95%, or from 80% to 99% may be achieved in certain embodiments of the apparatus and/or methods described herein. In some embodiments, a reduction in concentration of drug substance of 100%, 90%, 80%, 70%, 60%, 50%, 40%, 30%, 20% or 10% may be achieved in certain embodiments of the apparatus and/or methods described herein. The percentage reduction in drug substance may be calculated according to the equation [100×(Cᵢₙᵢₜ−C_{final})/Cᵢₙᵢₜ] wherein Cᵢₙᵢₜ=drug substance concentration prior to UV sanitising according to the methods described herein and C_{final}=drug substance concentration after UV sanitising according to the methods described herein. The reduction in concentration of drug substance as described in this paragraph may be at any suitable wavelength of UV light. In one embodiment, the reduction in concentration of drug substance in this paragraph is at 248 nm. In one embodiment, the reduction in concentration of drug substance in this paragraph is at 193 nm. In one embodiment, the reduction in concentration of drug substance in this paragraph is at 248 nm or 193 nm. In one embodiment, the reduction in concentration of drug substance in this paragraph is achieved using an excimer laser source.

It will be appreciated that different drug substances may require different doses of UV radiation in order to achieve a given reduction in concentration and that the UV may impact the drug substance in different ways depending on its molecular structure and UV susceptibility. In some embodiments, the dose response of a particular drug substance is determined prior to using the methods and/or apparatus described herein. In such embodiments, a dose response may be determined by correlating the concentration of a given drug substance remaining after UV irradiation as a function of energy of UV light in mJ/cm² at a given wavelength for a given surface (or type thereof).

In some embodiments, the energy delivered to a surface may be of a quantity sufficient to alter all susceptible drug substances present on that surface. In some embodiments, the energy delivered to a surface may be of a quantity sufficient to alter all susceptible illicit drug substances present on that surface. In some embodiments, the energy delivered by the light source is of a quantity sufficient to alter cocaine present on a given surface.

### Sanitising (Combinations)

In some embodiments. there is described herein a method for sanitising a surface of an object, comprising:
conveying the object through UV light from an excimer laser at a speed of greater than 1.0 m/s, such that the UV light irradiates the surface,
wherein the UV light has a wavelength, intensity and pulse duration sufficient to reduce a concentration of drug substance and reduce microorganisms and/or viruses on the surface.

In one embodiment, there is described a method for sanitising a surface of each of a plurality of objects, comprising:
conveying the objects through UV light from an excimer laser at a speed of greater than 1.0 m/s and at a frequency of greater than 10 Hz, such that the UV light irradiates the surface of each of the plurality of objects,
wherein the UV light has a wavelength, intensity and pulse duration sufficient to reduce a concentration of drug substance and/or reduce microorganisms and/or viruses on the surface of each of the plurality of objects.

In other embodiments, there is described herein an apparatus for sanitising a surface of one or more objects, comprising:
an excimer laser that emits pulses of UV light; and
a system that conveys the object(s) through the UV light at a speed of greater than 1.0 m/s, optionally between 1.0 m/s and 8.0 m/s;
wherein the UV light has a wavelength, intensity and pulse duration sufficient to reduce a concentration of a drug substance on the surface and/or reduce microorganisms and/or viruses on the surface.

### EMBODIMENTS

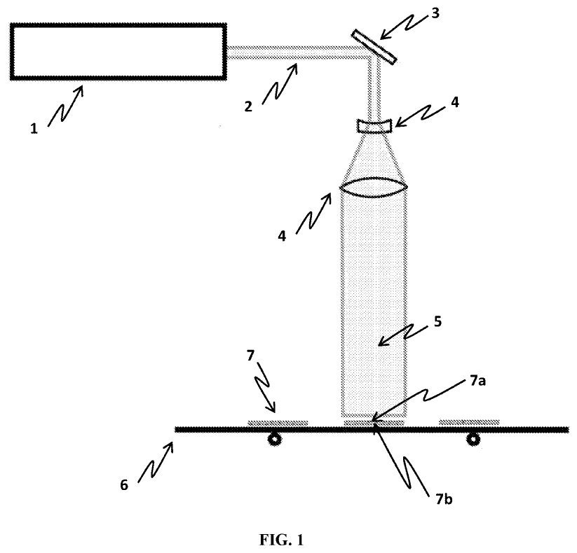

With reference to the embodiment depicted in FIG. 1, a laser 1 fires a laser beam 2, which is directed towards an object, depicted in this embodiment as a singularised banknote (“note”) 7, by a turning mirror 3 (additional turning mirror(s) may be used; not shown) and the laser beam is expanded to a desired size 5 by a beam expansion lens system depicted as beam expanding lenses 4 or a beam expanding telescope. The beam size 5 is selected to be slightly larger than the object 7. In the illustrated embodiment, the object is a banknote 7. A trigger system (not shown) detects the presence of the object on the object conveyer system, which in the illustrated embodiment is a singularised note transport conveyer 6, and commands the laser to fire at the appropriate time. The movement of the banknote is effectively frozen for the short laser pulse length, in some embodiments for approximately 15 ns.

The laser may be equipped with either a stable or unstable resonator (not shown) set to reduce beam divergence. If a stable resonator is used, the beam uniformity may be controlled by the use of a beam expanding telescope or by the use of crossed cylindrical homogenisers or insertable wedged plates (if the native homogeneity is insufficient). The uniformity of the expanded unstable resonator beam may be sufficient to eliminate the need for auxiliary beam homogenisation techniques and may also remove any positional sensitivity of the banknote to laser distance.

The embodiment depicted in FIG. 1 exposes a first face 7a of the object 7 to the incident laser beam 5. In such embodiments, a second opposing face 7b of the object 7 may be sanitised by turning or flipping the object (means for which are not shown) and passing it through the laser beam 5 a second time.

The embodiment depicted in FIG. 1 shows a horizontal (with respect to the ground) conveyer belt 6 for conveying the object 7 to the laser beam 5. However, the invention is not limited to receiving objects in this form. In other embodiments, the object may be conveyed vertically with respect to the ground, or may be circular around a drum, or may comprise any other suitable system or mechanism capable of passing objects through the incident laser beam.

The embodiment depicted in FIG. 1 has the laser beam 5 incident on the object at an angle of 90°. However, in other embodiments, different incident laser beam angles may be used.

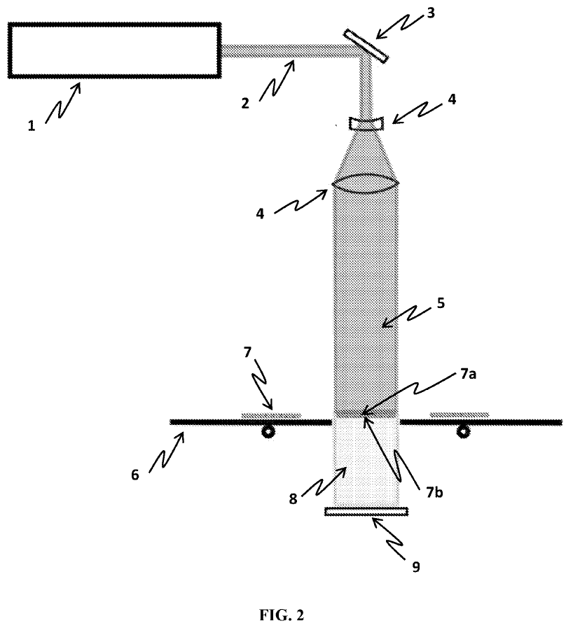

With reference to the embodiment shown in FIG. 2, the incident beam size 5 is modified (with respect to the embodiment shown in FIG. 1) to be twice the height of the note (in FIG. 2, running in a plane perpendicular to the plane of the page). Half the focal spot of the beam is incident on a first surface 7a of the object 7, and half the beam not coincident on the first surface of the object is instead incident on a retroreflecting mirror 9 positioned to direct the retroreflected beam 8 onto a second surface 7b of the object 7 opposing the first surface 7a. The retroreflecting mirror 9 may be slightly angled to direct the beam onto the second surface 7b. In some embodiments, the retroreflecting mirror 9 and laser beam 5 are relatively positioned such that a portion of the laser beam 5 is directly incident on the object 7, and the remaining portion is directly incident on the mirror 9. In other embodiments, the object conveyer system 6 comprises a conveyer made of a UV transparent material and a portion of the laser beam 5 is directly incident on the object, and the remaining portion passes through the conveyer made of a UV transparent material prior to being incident on the mirror 9. UV transparent materials may include polypropylene, silica, calcium or magnesium fluorides, or Teflon. In yet further embodiments, the object conveyer system 6 conveys the objects 7 across an illumination stage or plate (not shown), and the illumination stage or plate comprises a UV transparent material. In such embodiments, a portion of the laser beam 5 is directly incident on the object, and the remaining portion passes through the illumination stage or plate made of a UV transparent material prior to being incident on the mirror 9.

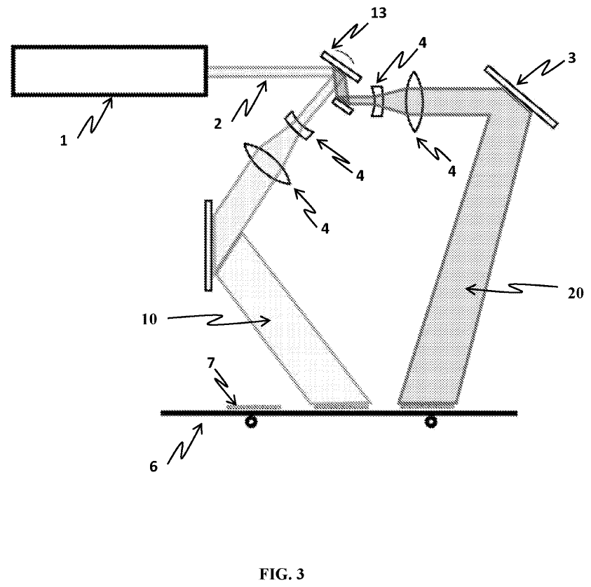

In the embodiment depicted in FIG. 2, two faces 7a and 7b of an object 7 can be irradiated simultaneously. It will be appreciated that a retroreflective mirror 9 may be used in conjunction with the embodiments depicted in FIGS. 3, 5 and 6 or their alternative embodiments as described herein.

With reference to FIG. 3, there is depicted an embodiment of an apparatus adapted to deliver two consecutive incident laser pulses from a single laser source 1 to a single surface of an object 7, shown as first laser pulse 10 and second laser pulse 20. In the embodiment of FIG. 3, a flipping mirror 13 (in one embodiment, a single axis galvanometer mirror) directs alternate pulses into each beam path sequentially. In use, an object 7 conveyed via the conveying mechanism 6 is first conveyed into the path of the first laser pulse 10, where it is irradiated, and after irradiation, is conveyed via the conveying mechanism 6 into the path of the second laser pulse 20.

In an alternative embodiment of the apparatus of FIG. 3, the flipping mirror is configured as a 2-dimensional galvanometer set so that the second pulse 20 is directed out of the initial plane of the object movement. In one embodiment, this may permit illuminating a second face of an object that has been turned downstream of the initial irradiation position and sent back to the laser. In this embodiment, the object 7 may be sent back to the laser to receive second pulse 20 on a conveyer system spaced apart, in a plane perpendicular to the plane of the page, from the conveyer system used to position the object in the beam of first pulse 10. In yet another embodiment, the first laser pulse 10 is configured to be directed towards a first surface of an object 7 and the second laser pulse 20 is configured to be directed towards a second surface of an object 7 opposing the first surface through use of a retroreflective mirror (not shown). In such embodiments, the retroreflective mirror may reflect the entire first or second laser pulse. Advantageously this permits a single laser beam to be directed towards two opposing faces of a flat or substantially flat object in a single object conveyer system.

In the embodiment of FIG. 3, the first laser beam 10 may be directed onto the object at a first angle and the second laser beam 20 may be directed onto the object at a second angle different to the first angle. In other embodiments, the flipping mirror 13 and/or turning mirror 3 may be adjusted such that the first and second laser beams (10 and 20) are incident on the object 7 at the same angle (i.e., the first and second angles are the same).

With reference to FIG. 4, there is depicted an alternative embodiment where laser beam 2 emitted from laser 1 is polarised through use of a polarising element 15 positioned, in some embodiments, upstream (with respect to the laser source) of the beam expansion lens 4. In some embodiments, the laser beam is split into S (25) and P (26) components and each component is delivered to the object 7 independently (as depicted in FIG. 4). In alternative embodiments, only the S (25) or only the P (26) component is delivered to the object 7. It will be appreciated that a polarising element 15 may be used in conjunction with the embodiments depicted in FIGS. 1-3 and 5-6 or their alternative embodiments as described herein.

In an alternative embodiment of the apparatus of FIG. 4, the polarisation element 15 is replaced with a beam splitting element (not shown) and two unpolarised components of the beam may be directed to the same object with a variable delay and optionally also a different incident angle. It will be appreciated that a splitting element may be used in conjunction with the embodiments depicted in FIGS. 1-3 and 5-6 or their alternative embodiments as described herein.

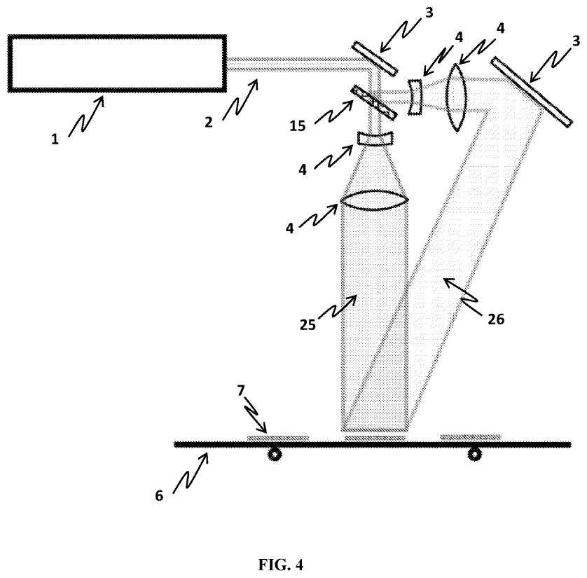

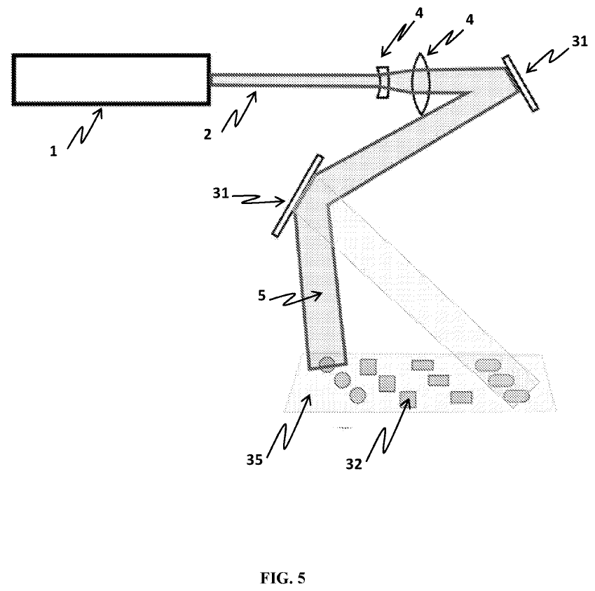

With reference to the embodiment in FIG. 5, there is depicted an apparatus adapted for sanitising objects 32 including coins, mail, parcels, boxes, packages, containers and/or bottles etc. The laser beam 2 from laser 1 may be delivered to a processing area 35 via a 2D galvanometer beam delivery system comprising scanning mirrors 31, resulting in beam 5. In some embodiments, an unstable resonator (not shown) with low beam divergence may be fitted in the laser. In such embodiments, the use of an unstable resonator in the laser with low beam divergence may eliminate the need for the traditional focussing lens in the galvanometer array, thereby increasing the delivery area, eliminating depth of field constraints and ensuring uniform illumination. In the embodiment depicted in FIG. 5, the objects 32 may be stationary or may be moving. In some embodiments, the objects 32 are conveyed by a conveying means (not shown). In some embodiments, the apparatus may comprise vision intelligence (not shown) so that object items are identified and illuminated for process optimisation. Advantageously, use of a laser beam 5 may allow a variety of non-uniform size and shape objects to be sanitised in a single process. Additionally, use of a laser beam 5 may allow scanning across areas of larger objects, such as parcels, boxes, packages, containers and/or bottles and thereby enable sanitising of complex surfaces.

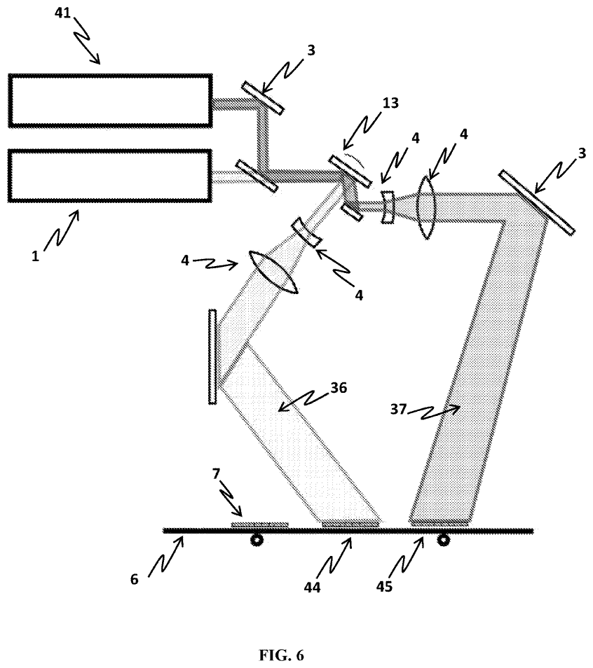

With reference to the embodiment in FIG. 6, there is depicted an apparatus comprising two laser sources, a first laser source 1 and a second laser source 41. The first laser source 1 emits a first UV laser beam and the second laser source 41 emits a second UV laser beam. In such an embodiment, the first and second laser beam pulses are alternately directed into two beam paths (36 and 37, respectively) by a flipping mirror 13. As depicted, the first pulse is directed onto a banknote 7 at a first position 44 by a turning mirror 3. As depicted, the second pulse is directed onto a banknote 7 at a second position 45 spaced apart from the first position 44 by a series of turning mirrors 3. In alternative embodiments, additional laser sources may be used. As described herein, the laser sources may emit light having different wavelengths, different beam intensities and/or different pulse durations. As described herein, there may be a delay between pulses.

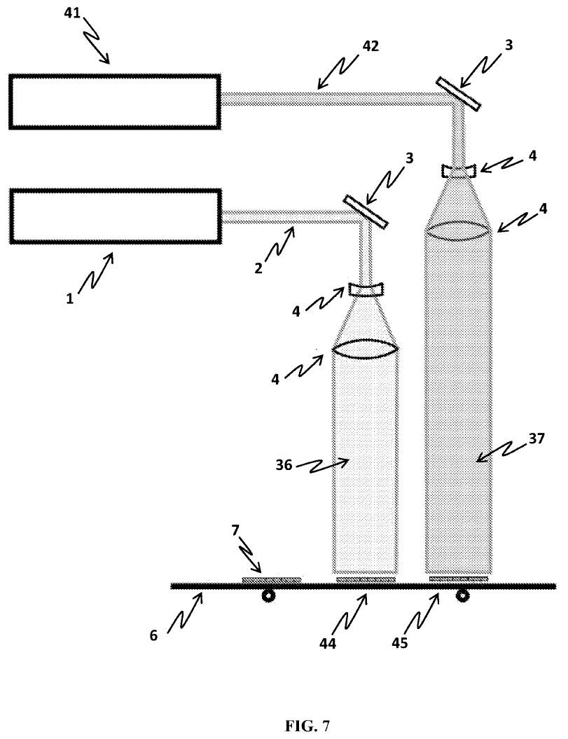

With reference to the embodiment in FIG. 7, there is depicted is a diagrammatic representation of a side view of an apparatus according to an embodiment of the present invention, where a first laser source 1 emits a first UV laser beam 2 and a second laser source 41 emits a second UV laser beam 42, wherein the first and second laser beam pulses are directed in two separate beam paths (36 and 37), where the first laser beam 36 is directed onto a banknote 7 at a first position 44, and the second laser beam 37 is directed onto a banknote 7 at a second position 45 spaced apart from the first position.

### Laser Parameters for Sanitising at Speed

Commercially available lasers can be divided into medium, high and very high power categories as per Table 1 below:

TABLE 1
Sanitising at speed parameters for different power lasers

| | | | | | | | | | Max. | Max. |
|---|---|---|---|---|---|---|---|---|---|---|
| | | | | | | | Object | Beam | energy | delivered |
| | Wave | | | | | Object | surface | delivery | delivered | energy |
| | length | Energy | Pulses/s | Power | Objects/s | speed* | area | efficiency | (/pulse) | (total) |
| Laser | nm | mJ | Hz - MAX | W | Hz | m/s | cm² | % | mJ/cm² | mJ/cm² |
| Medium | 248 | 600 | 100 | 60 | 33 | 4.29 | 100 | 80% | 4.8 | 14.5 |
| Power | 248 | 600 | 100 | 60 | 50 | 6.5 | 100 | 80% | 4.8 | 9.6 |
| | 248 | 500 | 200 | 100 | 33 | 4.29 | 100 | 80% | 4 | 24.2 |
| | 248 | 400 | 200 | 80 | 33 | 4.29 | 100 | 80% | 3.2 | 19.4 |
| | 193 | 200 | 100 | 20 | 33 | 4.29 | 100 | 80% | 1.6 | 4.8 |
| | 193 | 200 | 100 | 20 | 50 | 6.5 | 100 | 80% | 1.6 | 3.2 |
| | 193 | 200 | 200 | 40 | 33 | 4.29 | 100 | 80% | 1.6 | 9.7 |
| | 193 | 150 | 200 | 30 | 33 | 4.29 | 100 | 80% | 1.2 | 7.3 |
| High | 248 | 1000 | 300 | 300 | 33 | 4.29 | 100 | 80% | 8 | 72.7 |
| Power | 248 | 1000 | 300 | 300 | 50 | 6.5 | 100 | 80% | 8 | 48.0 |
| | 193 | 400 | 150 | 60 | 33 | 4.29 | 100 | 80% | 3.2 | 14.5 |
| Very | 248 | 1000 | 600 | 600 | 50 | 6.5 | 100 | 80% | 8 | 96.0 |
| High | 248 | 1000 | 600 | 600 | 33 | 4.29 | 100 | 80% | 8 | 145.5 |
| Power | 248 | 1000 | 600 | 600 | 50 | 6.5 | 100 | 80% | 8 | 96.0 |

*Indicative speed based on, for example, a banknote length dimension of ~13 cm, where banknotes are laid longways end to end without space between them, moving at the stated frequency. The skilled person can determine the indicative speed for other length objects accordingly. For example, the indicative speed based on a banknote length of 15 cm with a separation of 50 mm between notes would result in 10 m/second at 50 Hz.

As will be evident from Table 1, medium power and energy lasers (100 W/500 mJ/200 Hz) can exceed the herein determined sanitising energy density (4+mJ/cm² for 248 nm and 2.5+mJ/cm² for 193 nm) for microorganisms and viruses and (4+mJ/cm² for 248 nm and 2.5+mJ/cm² for 193 nm) for drug substances; hereinafter, “determined sanitising thresholds”) at 248 nm for single sided treatment by a factor of four for the fastest sorting machines (50 notes per second), thereby offering the chance to quadruple the dose on a single side or double the dose and transfer the extra energy to the reverse side of the note. Energy densities approximating 15 mJ/cm² could be reached with a two-laser solution—one laser per side of each banknote.

High power lasers (300 W/1000 mJ/300 Hz) enable the determined sanitising thresholds to be exceeded. They enable 8 mJ/cm² to be delivered in a single pulse at 248 nm, with up to 6 pulses being delivered to one side of a note at the highest throughput speeds, or could be split to deliver half of this power to each side of a note.

Very high-power lasers double the delivery capability of the high power units with up to 600 W of average power available. Multiplexing of these 600 W lasers delivers even higher powers. Average powers to 40 W are available at 193 nm from medium power lasers with this increasing to 60 W for the high power devices. Very high power lasers are not offered commercially at 193 nm.

The pulse repetition rate flexibility of the excimer laser enables the above energies to be consistently delivered to the target objects as the conveying machine moves from zero to full speed. For sorting systems operating at speeds to 33 Hz (that is, 33 notes per second), the energy densities delivered can be proportionally higher.

All of the above solutions can have the beam delivered to the full area of e.g. the banknote or sectioned with galvanometer beam guided tracking delivering higher fluences to parts of the banknote sequentially.

The excimer laser systems detailed above enable the processing of areas exceeding 5,000 cm² (0.5 m²) per second. The medium power excimer lasers detailed above enable the processing of areas exceeding 5,000 cm² (0.5 m²) per second with fluences higher than those exemplified. This processing area capability increases past 1 m² per second with the use of high power lasers and is even greater with the implementation of very high power excimer laser sources. This processing speed is orders of magnitude higher than that possible by other technologies in the same timeframe, such as lamp or LED. This processing speed makes the apparatus and/or methods described herein compatible with high speed conveying or sorting machinery used in the banknote and parcel transport industries.

All references cited herein, including patents, patent applications, publications, and databases, are hereby incorporated by reference in their entireties, whether previously specifically incorporated or not.

### EXAMPLES

The present invention will now be described with reference to the following examples which should be considered in all respects as illustrative and non-restrictive.

### Example 1—Laser Pulsed Light for Sanitising Banknotes

A microorganism or virus is applied to the surface of an object at a known concentration. The microorganism or virus is applied either in stock solution or in an organic matrix including bovine serum albumin, mucin and tryptone or following international standard ASTM E2197-17e1 (Standard quantitative disk carrier test method for determining bactericidal, virucidal, fungicidal, mycobactericidal, and sporicidal activities of chemicals, ASTM International, West Conshohocken, P A, 2017) and dried. In one experiment, the surface is a sterile Australian polymer banknote. The microorganism or virus may be a pathogenic coronavirus such as a human coronavirus (SARS-CoV-2), a mouse betacoronavirus MHV-CoV, or a model organism, for example MS2 or lambda bacteriophage. Samples are then stored in the dark in a humidity-controlled environment and under standard laboratory conditions (25° C., 1 atm). Control samples are isolated out of UV light. Experimental samples are exposed to a UV laser irradiation at a known wavelength, intensity and pulse duration, with the resultant energy incident on the surface (in J/cm²) calculated by multiplying the intensity (in W/cm²) by the pulse duration (in seconds). Swabs are taken from the surface immediately prior to UV irradiation and immediately after irradiation and the reduction in number of a population of a particular microorganism or virus is calculated according to Equation 1 or Equation 2:

percent reduction=[(N₀−Nₜ)×100]/N₀ Equation 1

log reduction=log₁₀(N₀/Nₜ) Equation 2

where N₀ is the number of viable microorganisms or viruses before UV irradiation and Nₜ is the number of viable microorganisms or viruses after UV irradiation.

A process such as described in Riddell et al. (2020) Virol. J. 17, 145 may be used.

The parameters for a process are given in Table 2.

TABLE 2
Tabulation of experiment parameters
for a specific microorganism or virus

| | Laser | | |
|---|---|---|---|
| | wavelength | | No. of |
| Sample # | (nm) | Incident energy (mJ/cm²) | pulses |
| Cotton banknote | 193 | 0.1, 1.0, 2.5, 5.0, 7.5, 10, 50 | 1 |
| Cotton banknote | 248 | 0.1, 1.0, 2.5, 5.0, 7.5, 10, 50 | 1 |
| Cotton banknote | 254 | 0.1, 1.0, 2.5, 5.0, 7.5, 10, 50 | 1 |
| Polymer banknote | 193 | 0.1, 1.0, 2.5, 5.0, 7.5, 10, 50 | 1 |
| Polymer banknote | 248 | 0.1, 1.0, 2.5, 5.0, 7.5, 10, 50 | 1 |
| Polymer banknote | 254 | 0.1, 1.0, 2.5, 5.0, 7.5, 10, 50 | 1 |

The reduction (% or log) in microorganism or virus is then plotted as a function of incident energy.

### Example 2—Sanitising Banknotes with Microbes

A series of experiments using excimer laser pulses at 248 nm and 193 nm were conducted utilising the model organism bacteriophage lambda and its host Escherichia coli. E. coli was also assessed independently as a model for bacterial contamination.

### Methods

### Viral and Bacterial Culture

Freeze-dried lambda bacteriophage (ATCC 23724-B2) and E. coli (ATCC 23724) were purchased and cultured as per ATCC instructions in LB broth/agar (E. coli culture) and TNT broth/agar (lambda bacteriophage in conjunction with host E. coli). Glycerol stocks (10% in LB) of E. coli were generated by culturing single colonies of E. coli overnight in a shaking incubator (37° C.; ˜250 rpm) to log phase (OD₆₃₀=0.5-0.6) before adding glycerol and storing at ˜20° C. No difference was observed between the utilisation of freshly cultured log-phase E. coli and frozen stocks for the independent E. coli sanitising experiments, likely due to the high stress of the microbe being dried onto the banknote. Therefore, glycerol stocks were directly diluted and applied to the banknotes for these experiments. However, fresh overnight E. coli culture were grown to log phase for each lambda experiment to ensure efficient viral infection of the E. coli host and clear plaque formation.

High titre lambda bacteriophage stocks were generated by viral propagation in E. coli following standard double agar overlay techniques. Briefly, fresh overnight cultures of E. coli were grown to log phase, added to molten TNP top agar (0.5% agar; 45° C.) and immediately poured onto pre-warmed (37° C.) TNT agar plates. Once the top agar had completely solidified, lower titre lambda bacteriophage stock was poured over the plate and incubated for 24 h (37° C.) until confluent bacterial lysis was observed. 5 mL of TNT broth was then added, the plates incubated for 20 min and the top agar/broth mix scraped off the plate and centrifuged at 4000 rpm for 25 minutes. The supernatant was collected and filtered through a 45 μm filter to remove any remaining bacteria and agar debris before storage at 4° C.

### Enumeration of Viral and Bacterial Samples

Enumeration of microbial stocks and viable microbes remaining post excimer laser treatment was conducted using the spot-titre method to count plaque forming units (PFUs; lambda) or colony forming units (CFUs; E. coli). Briefly, 10-fold serial dilutions were made in media and spotted onto an appropriate media plate (LB agar for E. coli; double agar overlay of TNT agar with E. coli host as described above for lambda). Agar plates were cultured for 24 h at 37° C. and the appropriate dilution determined and counted for each sample to calculate concentration.

Once individual microbial loads were determined, the data were transformed to PFU or CFU per mm²+1 before the logarithm was taken. This permitted the inclusion of individual samples where microbial levels had been reduced to undetectable levels in the pooled analyses (since log₁₀(1)=0 whereas log₁₀(0) is undefined).

### Banknotes

All banknotes assessed in this study were new Australian polymer banknotes. Banknotes were sanitised immediately prior to use via UV-C irradiation (15 min per side, UV-C LED) and then handled aseptically throughout the experiment. Microbial stocks were then diluted as required in media before application to the notes in 1 μL spots, with the contaminated area measured and determined to consistently be ˜2.4 mm²/spot. For experiments at 248 nm, 1 cm² cassettes were cut and placed into the base of sterile 24-well tissue culture plates before application of the microbe in 1 μL to the centre of the cassette for automated laser firing. For 193 nm, 1 μL spots were applied to marked locations and laser firing was targeted and triggered manually. Spots were then reconstituted in set volumes of appropriate media broth and microbial enumeration conducted via spot titre assays as described.

Control samples were treated identically to test samples with the exception of laser firing, hence they were also applied to the banknotes in conjunction with the test samples and allowed to dry. Due to the variable levels of cell death/viral inactivation that resulted from the drying process being influenced by factors such as drying time, temperature, and time before processing, the starting level of contamination was considered to be the level of contamination remaining in the control samples after treatment, rather than the amount applied to the note before drying and laser treatment. This was required to permit direct determination of the effect of only the laser pulse(s) on microbial survival, but also means that the level of microbial contamination at the time of laser firing may have been underestimated, even though samples were processed as quickly as possible post sanitising.

### Laser

ATL excimer lasers with output energies to 15 mJ were utilised to test the efficiency of banknote sanitising over small areas. Laser energy at the sample was directly measured with the use of a power meter (calibrated Ophir PE-50-PF-C head and Laserstar readout) and the power measured whenever the fluence per pulse was adjusted to ensure laser power was consistent across experiments. All fluences above 4 mJ/cm² for 248 nm and 2.5 mJ/cm² for 193 nm were delivered as multiple pulses at a repetition rate of 10 Hz of 4 mJ/cm² and 2.5 mJ/cm² respectively, thus 8 mJ/cm² was two consecutive 4 mJ/cm² pulses and so forth. For fluences below 4 mJ/cm² at 248 nm, an attenuator (Optec AT-4020) was used to reduce power from a 4 mJ/cm² pulse such that the power at the target was at the desired level. The sanitising treatment was therefore delivered in 0.1-0.8 seconds, although notably, far higher speeds could have been easily achieved, with pulse repetition rates of up to 500 Hz being readily within the capabilities of the laser used.

### Statistical Analyses

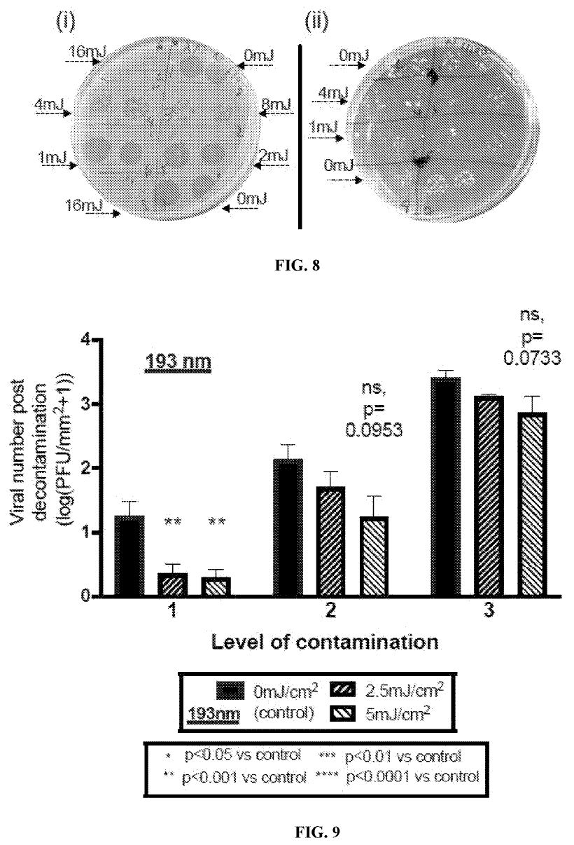

Statistical analyses were conducted for each microbial contamination level separately as the varying sample size and fluences assessed did not permit a two-way ANOVA. Therefore, individual one-way ANOVAs were run, each followed by a Dunnett's multiple comparison test comparing the means of each laser treatment with the mean of the control 0 mJ/cm² column. The only exceptions were for the lowest viral contamination level and decontamination of bacteria on plastic surfaces, for which only two groups (control and 4 mJ/cm²) were assessed, thus an unpaired, two-tailed t-test was run for those data sets only. All statistically significant (p<0.05) results are marked as appropriate in FIGS. 8 to 14.

### Sample Size

Sample size varied across contamination levels and fluence. For lambda bacteriophage at 248 nm, data for 251 individual samples were pooled from 4 independent experiments, totalling n=32-48 per fluence for all contamination levels combined. Similarly, data for 40 individual samples were pooled from 3 independent lambda experiments at 193 nm; combined n=12-14 per fluence. For E. coli at 248 nm, data for 250 individual samples were pooled from 9 independent experiments for a combined n=54-78 per fluence for 0, 1 and 4 mJ/cm² and n=12-16 per fluence for 2, 8, 16 and 32 mJ/cm². At 193 nm, data for 65 individual E. coli samples were pooled from 3 independent experiments for a combined total n=20-23 per fluence. Finally, preliminary data on plastic surfaces assessed data from 1 experiment, with n=9 for both control and 4 mJ/cm² treated groups.

### Results

Representative images of spot-titre agar plates enumerating lambda bacteriophage plaques and E. coli colonies post sanitising are shown in FIGS. 8 (i) and (ii), respectively. The results show a significant decrease in target numbers of microbes on banknotes at energies of 4+mJ/cm² for 248 nm and 2.5+mJ/cm² for 193 nm.

Effective viral sanitising of lambda bacteriophage was achieved with 248 nm pulses over a wide range of contamination levels from approximately 1 PFU/mm² to 104 PFU/mm² (FIG. 10). Increased laser fluence was clearly correlated to increased efficacy, especially at higher viral loads (FIG. 8(i), FIG. 10). In particular, 16 mJ/cm² was consistently more effective than 1-8 mJ/cm² and removed ˜1.5-2 logs of contamination even at very high viral loads. 32 mJ/cm² resulted in even higher levels of sanitising of ˜3 logs at higher contamination levels. Nonetheless, at the lower assessed levels, 4 mJ/cm² was highly effective. Viral contamination was also readily removed by laser pulses at 193 nm, with significant reductions at both 2.5 and 5 mJ/cm² at the lowest assessed viral load, approximately 10 PFU/cm² (FIG. 9). The viral number was not significantly altered at the two higher contamination levels assessed, however, the trend of the data was consistent and the sample size was small for these two cohorts (n=3-4), thus statistical tests may have thus lacked sufficient power to achieve significance. In any case, it is evident that excimer laser pulses provided effective and rapid viral sanitising in less than one second.

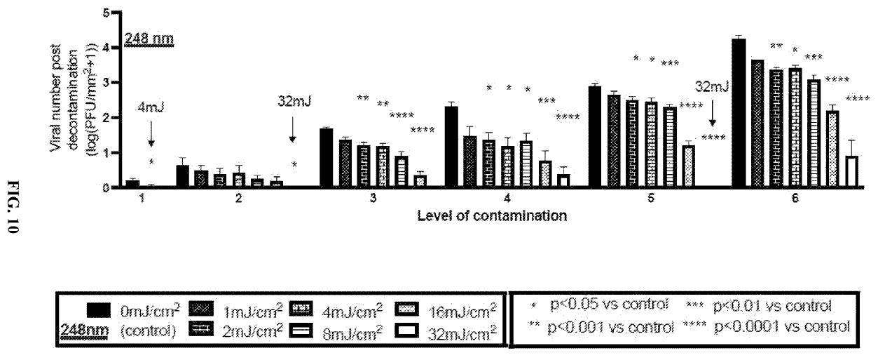

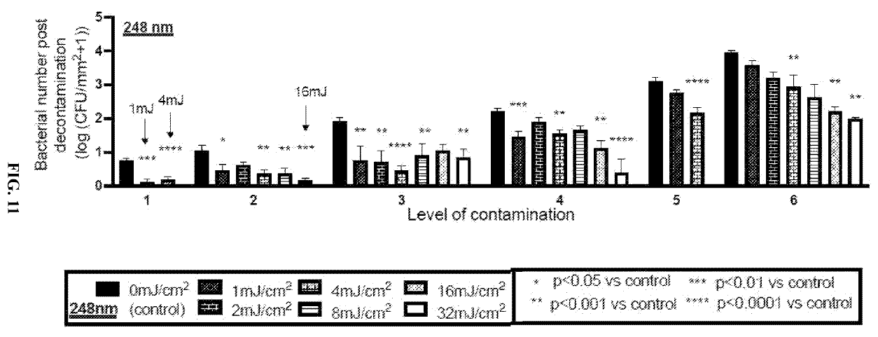

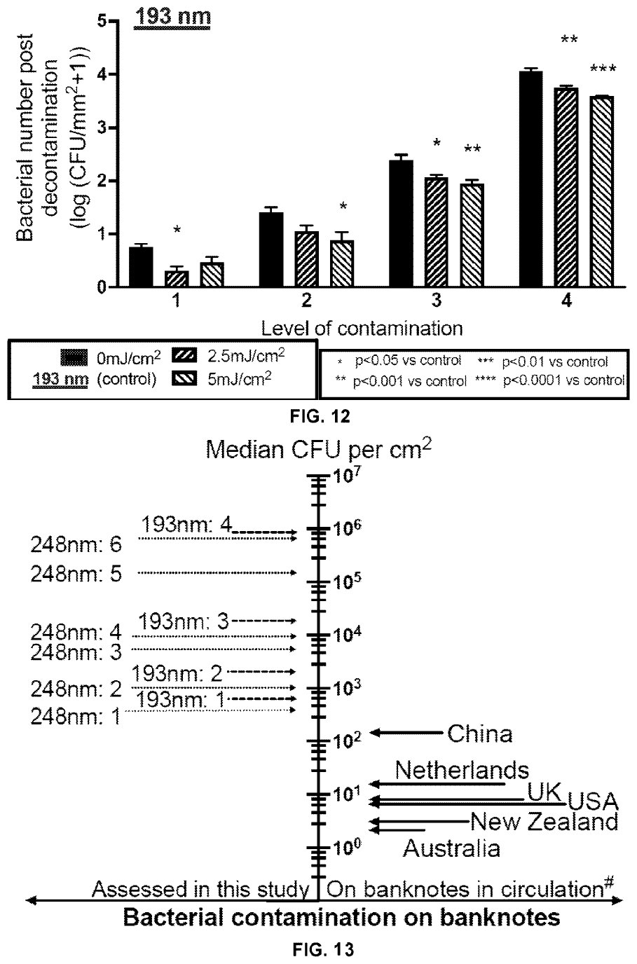

Furthermore, similar sanitising efficacy was seen for banknotes contaminated with E. coli, demonstrating that excimer laser pulses at both 248 nm (FIGS. 11 and 193 nm (FIG. 12), could also successfully remove bacterial contamination from banknotes over a wide range of contamination levels. Consistent with the viral data, increased bacterial sanitising efficacy was observed with increased laser fluence, particularly at higher bacterial contamination levels, however, at the lowest assessed contamination levels, 1-4 mJ/cm² had substantial sanitising efficacy.

Considering that the lowest bacterial contamination levels assessed in this study were ˜100× the median contamination levels reported on Australian polymer banknotes in circulation (FIG. 13; circulating contamination data summarised from Vriesekoop et al. (2010) Foodborne Pathogens and Disease, 7, 1497-1502), this indicated that at typical contamination levels, higher laser fluences may not be required for effective sanitising. However, individual notes may have far higher levels of microbial contamination, with one study, for example, reporting that the numbers of bacteria isolated from individual US currency ranged from 2×10¹ CFU/cm² to 2.5×10⁴ CFU/cm². Nonetheless, those levels were still well below the highest contamination levels assessed in this study. Accordingly, effective sanitising of even the most contaminated banknotes can be conducted using an excimer laser in accordance with the methods and apparatus described herein at very fast speeds, such as those speeds currently utilised in banknote sorting machines.

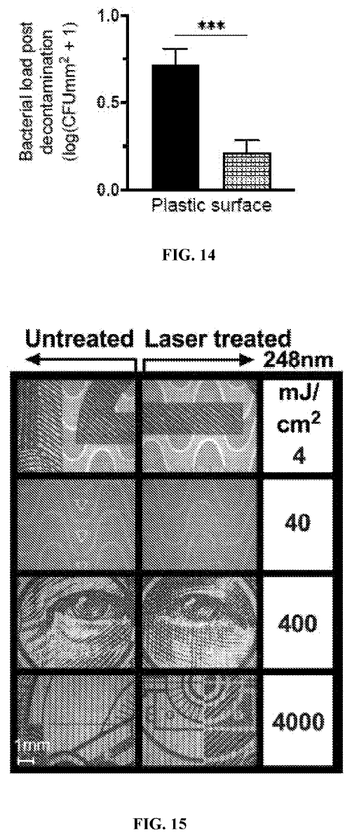

Preliminary data also demonstrated that efficient microbial killing was also observed on other surfaces, with similar efficacy seen during laser sanitising of E. coli on plastic surfaces (FIG. 14), supporting more broad applications of excimer laser sanitising using the methods and/or apparatus described herein.

Viral loads on banknotes in circulation have not been well established, however, Harcourt et al., 2020, PLOS Negl Trop Dis; 14(11): e0008831 demonstrated that infectious SARS-CoV-2 remained detectable on some US bank notes for up to 7 days in cool laboratory conditions (4° C.) at starting viral loads of ˜5×10³ PFU/mm², viral loads consistent with the higher contamination levels we assessed. Another study, Riddell et al., 2020, showed stability of SARS-CoV-2 on banknotes for up to 28 days from starting contamination levels of ˜2.3×10⁵ PFU, approximately half of the highest lambda dose we assessed, however, the area over which the dose was applied was not reported. Nonetheless, the two studies collectively indicate that the viral loads of the model virus at which we have demonstrated efficient excimer laser sanitising in under 1 second are likely to be equivalent to, or higher than, the levels at which SARS-CoV-2 can persist for weeks on banknotes.

No damage was observed on the banknotes at 248 nm at laser fluences of between 4-4000 mJ/cm² (FIG. 15). Matched untreated and laser treated areas shown in FIG. 15 clearly demonstrate that the laser sanitising did not damage or alter the banknote in any discernible way, even at 40× magnification following treatment with 4000 mJ/cm² at 248 nm, which is 125×higher than the highest fluence assessed for sanitising purposes in this study. Laser treatment at 193 nm similarly caused no discernible damage or alteration of any kind to the banknotes.

In summary, the data revealed that excimer laser pulses at both 248 nm and 193 nm efficiently and rapidly removed microbial contamination from banknotes. Preliminary data also demonstrated that efficient microbial killing was also observed on other surfaces. In contrast to existing methods, excimer laser sanitising can be conducted extremely quickly, in a fraction of a second, permitting sanitising at the very high speeds currently used during commercial sorting of banknotes. Furthermore, in contrast to many potential sanitising methods, the laser pulses did not damage the note in any discernible way.

### Example 3—Sanitising Banknotes with Drug Substances

### Methods

### Preparation of Drug Stocks

A series of experiments were conducted utilising pharmaceutical formulations of hyoscine butylbromide (HBB; Buscopan Forte tablets containing 20 mg HBB/tablet) and dexamphetamine (DEX; Aspen Dexamfetamine tablets containing 5 mg dexamphetamine sulfate/tablet). HBB and DEX were utilised as model compounds to assess removal of illicit drugs from banknotes as although their interactions with the human brain differ substantially, they share similar chemical structures with cocaine and methamphetamine respectively as shown below:

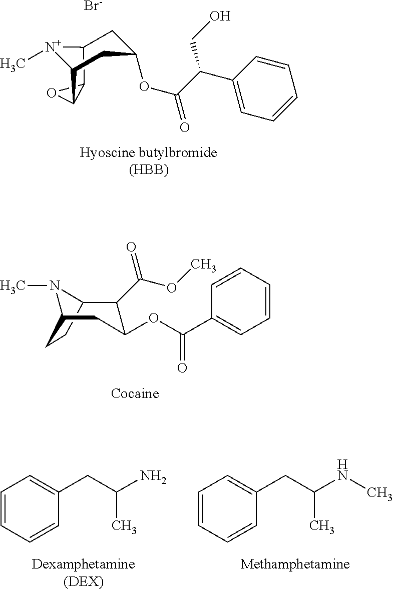

Tablets were purchased from a local pharmacy and dissolved with agitation to 1 mg/mL in distilled and deionised water. Experiments were conducted with both filtered (45 μm filter to remove non-water soluble components) and non-filtered samples as stated. Both HBB and DEX are soluble in water at concentrations far higher than 1 mg/mL, thus all drug was presumed dissolved and concentration of stocks and all subsequent dilutions calculated by division of the amount of active drug/tablet over the volume of water in which it had been dissolved, accounting for any subsequent dilutions by volume. Fresh 1 mg/mL drug stocks were made daily and were then diluted as required in distilled and deionised water.

### Assessment of Drug Concentration

Drug concentration was quantified via two methods. Firstly, the well-established and non-specific oxidation of the drug and coincident reduction of purple coloured potassium permanganate (KMnO₄) to green MnO₄ manganate ions under alkaline conditions. Assessment of the kinetics of the colorimetric reaction and comparison to a standard curve of drug concentrations permits interpolation of the concentration of the drug in an unknown solution from the absorbance. Maximal absorbance of the product is at ˜610 nm, but the absorbance peak is broad, permitting spectrophotometric assessment over a range of wavelengths.

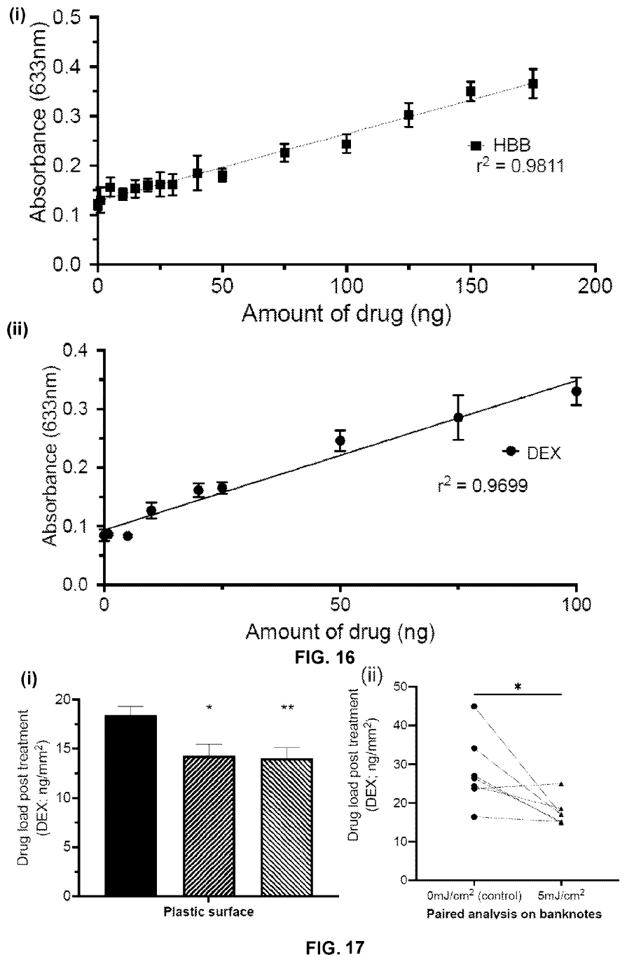

The kinetics of the reaction with HBB were therefore assessed and it was determined that consistent with previous studies, levels of HBB could be quantified spectrophotometrically at 633 nm 15 minutes after reacting with 2.5×10⁻¹ M NaOH (Sigma Aldrich) and 5×10⁻³ M KMnO₄ (Sigma Aldrich). To provide sufficient sensitivity at the contamination levels applied to the note, reaction concentrations were then maintained while total reaction volume was decreased over 20-fold to 40 μL total, comprising 20 μL reaction mix and the test sample reconstituted in 20 μL of distilled and deionised water. The kinetics of the miniature reaction were then assessed, with 10-fold dilution in distilled and deionised water immediately prior to spectrophotometric assessment to ensure the sample had sufficient volume for analysis. The data revealed the concentrations over which the Beer Lambert law was obeyed and absorbance was linearly related to concentration successfully decreased from the low microgram to low nanogram range. The pooled HBB standard curve is shown is FIG. 16(i), in addition to the linear regression analysis.

Quantification of DEX with KMnO₄ had not been reported in the literature, however, numerous other drugs had been quantified via that method and it had been reported that DEX did react with KMnO₄, with the kinetics of the reaction being very rapid. We therefore assessed the reaction with DEX, determining that the reaction was indeed very rapid, occurring within 5 minutes. The sensitivity of the assay was even higher for DEX than for HBB, with the linear relationship continuing to a limit of detection of approximately 10 nmol/reaction (FIG. 16(ii)).

### Banknotes

All banknotes utilised were new Australian polymer banknotes and were prepared as described for the microbial decontamination studies, with the exception of sterilisation before treatment and working under aseptic conditions not being necessary for the drug studies. 1 μL of drug at the desired concentration was applied in spots as previously described for 248 nm and 193 nm experiments and allowed to dry, with the resulting drug contamination covering approximately 1.23 mm² for filtered samples and approximately 2.4 mm² for non-filtered samples. Laser decontamination was also conducted as previously described for microbial analyses before reconstitution of the remaining drug in distilled and deionised water and assessment of the concentration.

The data revealed that at the very high sensitivity levels now achieved in the assays, there was considerable and highly variable interference of banknote ink on the kinetics of the oxidation/reduction reaction with alkaline KMnO₄. Unfortunately, this meant that contaminated spots could not be placed randomly on the banknote and then assessed, as had been conducted for microbial sanitising tests. However, since it was determined that the interference with the colorimetric reaction occurred in the presence of the drug regardless of whether laser treatment had been performed and was thus independent of the variable of interest, the sanitising mediated by that laser treatment, it was still possible to circumvent the problem via precisely conducted paired analyses. 1 μL of drug at a set concentration was spotted at precisely the same locations on two or more identical banknotes. Laser sanitising treatment was then conducted, the colorimetric reaction performed as previously described and paired statistical analyses conducted to compare the interpolated drug doses between control and test samples at each position. Therefore, the overall effectiveness of the sanitising effect could still be assessed qualitatively by paired analyses comparing the resulting interpolated drug load in the treated versus the control sample at the exact same location. Quantitation of the exact amount of drug removed was nonetheless conducted on a polystyrene surface and a second method of detection via commercial amphetamine tests also utilised to independently confirm sanitising was occurring.

### Commercially Available Amphetamine Detection Tests

Saliva (detection limit of 50 ng/mL amphetamine) and urine (detection limit of 300 ng/mL amphetamine) testing kits were purchased from MediNat Australia. The minimum testing volumes were determined to be ˜2.5 mL for the saliva test and ˜200 μL for the urine detection test. Testing kit instructions were followed for assessment with the obvious exception of the type of sample being assessed and the volumes being minimised to increase the sensitivity.

### Sample Size

For assessment of drug sanitising via colorimetric methods after laser treatment at 193 nm, 43 HBB samples were tested on banknotes for a total of n=12-19 per fluence; data were pooled from 2 independent experiments. 28 of those 43 were paired analyses. 14 samples of DEX were also assessed via paired analysis on banknotes and a further 73 assessed on plastic (polystyrene) surfaces; n=22-28 per 193 nm fluence; data were pooled from 3 independent experiments. Drug sanitising at 248 nm assessed 7 HBB samples (n=3-4) and 8 DEX samples (n=4) colorimetrically; data is from 1 experiment. Additionally, analyses of DEX contamination with commercial detection kits were conducted on 2-3 samples per fluence for the 248 nm data shown in Table 3. Data for 193 nm is preliminary with only 1 sample assessed per fluence, however, that was a pooled sample from four individual test spots/fluence.

TABLE 3
Summary of DEX saliva (S) and urine (U) test data for which control 0
mJ/cm² samples were positive and laser-treated samples were negative

| | | | Minimum | | | |
|---|---|---|---|---|---|---|
| Laser | Laser | | amount removed | | Minimum amount | |
| wavelength | fluence | Dose | to be below LOD | Dose/note | removed per note | |
| (nm) | (mJ/cm²) | (μg DEX/mm²) | (DEX ng/mm²) | (μg DEX) | (DEX μg/200 cm²) | Test |
| 248 | 4 | 63 | 11 | 1263 | 221 | S |
| 248 | 4 | 19 | 9 | 379 | 170 | S |
| 248 | 4 | 8 | 7 | 167 | 149 | S |
| 193 | 5 | 16.3 | 4.1 | 326 | 82 | U |
| 193 | 10 | 16.3 | 4.1 | 326 | 82 | U |
| 193 | 2.5 | 16.3 | 4.1 | 326 | 82 | U |

Limit of Detection

The minimum amount of drug required to be removed (ng/mm²) in order to be below the limit of detection (LOD) was calculated mathematically via a series of calculations as follows: M=the stated manufacturers stated specifications for the kit's LOD (ng amphetamine/mL); =300 ng/mL for urine tests and 50 ng/mL for saliva tests; V=final volume of water the samples were tested in (mL); A=area covered by starting amount of drug applied to note (mm²); O=the starting amount of drug applied to the banknote (ng); =concentration of stock drug solution (calculated as previously described)×volume applied to banknote.

The loss of drug due to the drying and reconstituting process was presumed to be minimal based on observations for dried vs. non-dried standard concentrations made during optimisation of the colorimetric assays.

$$LOD of experiment = the minimum amount of DEX ( ng ) required for a positive result was calculated = {manufacturer}^{′} s specifications \times volume used in the experiment = M \times V \qquad ( 1 )$$

$$X = minimum amount of drug required to be removed ( ng / {mm}^{2} ) by decontamination to have a positive control sample and a negaive laser treated sample = \frac{\begin{matrix}starting amount of drug - \\ minimum amount of drug required for positive test\end{matrix}}{area of banknote contaminated by drug} = \frac{O - LOD}{A} \qquad ( 2 )$$

Therefore, the laser treatment would need to remove at least X ng/mm² to give a positive result in the untreated control and a negative result in laser test sample. The commercial tests only gave binary results (that the drug was detected or not) and thus that the amount of drug was higher or lower the LOD respectively. Accordingly, it is possible that the laser decontamination could have removed substantially more drug from the banknotes than the minimum amount reported, but the degree was not quantifiable via this method. Thus, only the minimum level of decontamination required are reported and discussed.

### Statistical Analyses

Statistical analyses were primarily conducted as described for microbial sanitising experiments, with one-way ANOVA's followed by Dunnett's multiple comparisons test to compare the mean of every group with the control group. Additionally, regression analyses were performed on the standard curves of absorbance versus HBB/DEX drug concentration to directly assess the linearity of the relationship. Finally, paired analyses on banknotes were assessed via paired two-tailed t tests between control and 5 mJ/cm² samples.

### Results and Discussion

A significant reduction of approximately 20-25% in the level of DEX contamination on a sterile polystyrene petri dish surface was achieved with laser treatment at 193 nm at fluences of 2.5-5 mJ/cm² when quantified via colorimetric reaction with alkaline KMnO₄ (FIG. 17(i)).

Paired analyses of DEX applied directly to banknotes confirmed that excimer laser pulses at 5 mJ/cm² were capable of significant sanitising of amphetamines on banknote surfaces (FIG. 17(ii)), although, as discussed, due to the altered reaction kinetics by the banknote ink, the magnitude of the reduction could not be accurately quantified by this method.

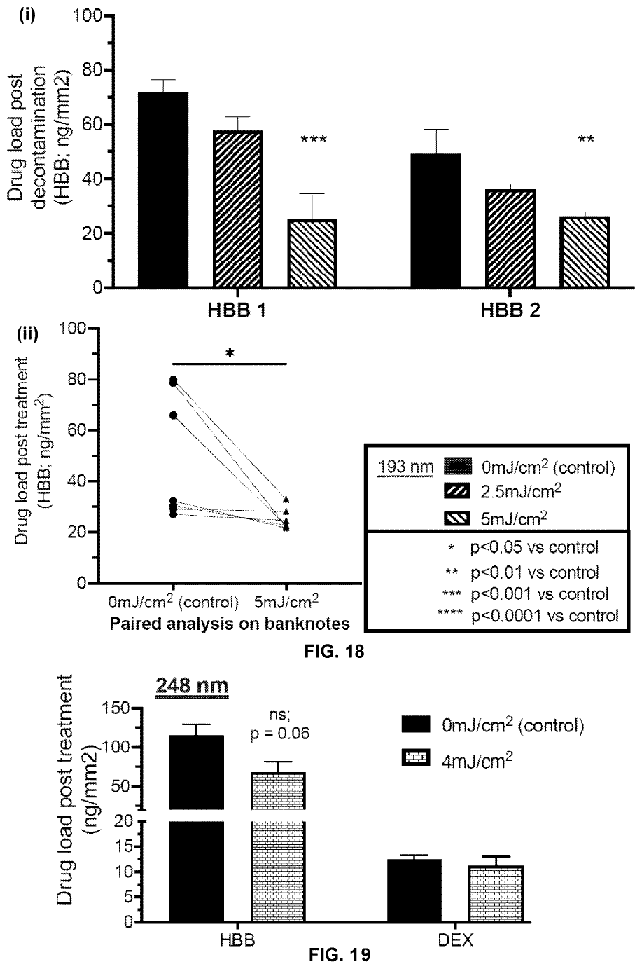

The successful reduction in the levels of HBB, a tropane alkaloid with a similar chemical structure to the tropane alkaloid cocaine, was also demonstrated directly on banknotes (FIG. 18(i)). Interestingly, it is possible that the interference of the banknote ink with the oxidation/reduction reaction of KMnO₄ was lesser than that observed with DEX, potentially due to the higher limit of detection. Regardless, similar results were observed in HBB1, in which the data were not paired and HBB2, in which all data points were paired. The paired analysis of control versus 5 mJ/cm² samples is incorporated into the data presented in HBB2 (FIG. 18(i)) but has been shown and analysed separately in FIG. 18(ii) to verify that consistent reductions in drug level are occurring after 193 nm laser sanitising regardless of the influence of any confounding independent variables.

Sanitising efficacy for both HBB and DEX was also conducted at 248 nm, however, the colorimetric analyses were not statistically significant (FIG. 19), although this may have simply been a result of the small sample size. Data suggests that laser pulses at 193 nm may be more considerably more effective at removing drugs from banknotes than 248 nm.

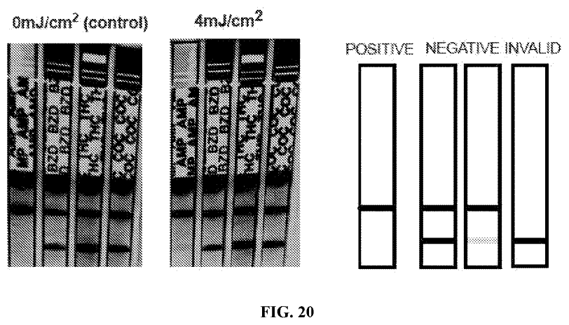

Nonetheless, the alternative DEX detection method utilised, the commercially available drug tests demonstrated that both 193 nm and 248 nm decreased the levels of DEX contamination on banknotes. An example of the results at 63 ng DEX/mm² on a banknote in a control untreated sample on a banknote and a sample decontaminated by a 248 nm laser pulse at 4 mJ/cm² is shown in FIG. 20 alongside the kit instructions for reading the results. It is important to note that the tests only indicate whether the drug is present above the minimum detection level or not, rather than quantifying the level of drug. Nonetheless, a series of urine and saliva testing kits close to the limit of detection clearly demonstrated that consistent with the colorimetric data on both plastic and banknotes, excimer laser pulses reduced the drug decontamination on notes (Table 3 above). Furthermore, the testing kits clearly demonstrated a small but consistent reduction in DEX contamination at 4 mJ/cm², even at extremely high contamination levels equivalent to ˜1.2 mg drug over both sides of a banknote.

Furthermore, whilst the observed reductions were small in magnitude per mm² (˜4-4.3 ng/mm² DEX on plastic surfaces and 4-12 ng/mm² DEX on banknotes assessed via the commercial tests), if replicated across the entire area of a banknote, which is ˜100 cm²/side it would correspond to a reduction of up to ˜40-120 μg per side or 80-240 μg per note. Determining the proportion of notes which would have similar, or higher, levels of drug contamination is difficult. The level of drugs detected on banknotes varies widely, with levels per note ranging from less than 0.1 ng to just over 1 mg, although the latter is extremely rare. Nonetheless, one review article suggested that based on their meta-analysis of currencies worldwide, levels of 100 ng/note indicated high contamination, yet another study of American notes found median contamination of ˜1.5 μg/note and, due to skewing by a few highly contaminated notes, mean contamination of ˜30 μg/note. Whilst the varying levels of contamination and different assessment methods in studies have therefore made establishment of the mean contamination of currency very difficult, it is evident that that levels of 100 μg drug/note are only found rarely on notes that are highly contaminated.

Therefore, it is clear that excimer laser pulses are capable of chemically altering or destroying levels of drug substances with similar chemical structures that, despite differences in molecular weight, would correspond to a reduction in contamination even at the highest levels of drug contamination reported on banknotes in circulation.

In summary, the data revealed that excimer laser pulses at both 248 nm and 193 nm efficiently and rapidly removed drug substance contamination.

Although the invention has been described with reference to specific examples, it will be appreciated by those skilled in the art that the invention may be embodied in many other forms in particular features of any one of the various described examples may be provided in any combination in any of the other described examples.

1. A method for sanitising a surface of an object, comprising:
conveying the object through UV light from an excimer laser at a speed of greater than 1.0 m/s, such that the UV light irradiates the surface,
wherein the UV light has a wavelength, intensity and pulse duration sufficient to reduce a concentration of drug substance on the surface.

2. The method of claim 1, wherein the method is for sanitising a surface of each of a plurality of objects, and comprises:
conveying the objects through UV light from an excimer laser at a speed of greater than 1.0 m/s and at a frequency of greater than 5 Hz or greater than 10 Hz, such that the UV light irradiates the surface of each of the plurality of objects,
wherein the UV light has a wavelength, intensity and pulse duration sufficient to reduce a concentration of drug substance on the surface of each of the plurality of objects.

3. The method of claim 2, wherein the objects are conveyed through the UV light at a frequency of between 5 Hz and 50 Hz or between 10 Hz and 50 Hz.

4. The method of any one of claims 1 to 3, wherein the UV light has a wavelength, intensity and pulse duration sufficient to chemically alter or destroy the drug substance on the surface.

5. The method of any one of claims 1 to 4, wherein the drug substance is cocaine.

6. A method for sanitising a surface of an object, comprising:
conveying the object through UV light from an excimer laser at a speed of greater than 1.0 m/s, such that the UV light irradiates the surface,
wherein the UV light has a wavelength, intensity and pulse duration sufficient to reduce microorganisms and/or viruses on the surface.

7. The method of claim 6, wherein the method is for sanitising a surface of each of a plurality of objects, and comprises:
conveying the objects through UV light from an excimer laser at a speed of greater than 1.0 m/s and at a frequency of greater than 5 Hz or greater than 10 Hz, such that the UV light irradiates the surface of each of the plurality of objects,
wherein the UV light has a wavelength, intensity and pulse duration sufficient to reduce microorganisms and/or viruses on the surface of each of the plurality of objects.

8. The method of claim 7, wherein the objects are conveyed through the UV light at a frequency of between 5 Hz and 50 Hz or between 10 Hz and 50 Hz.

9. The method of any one of claims 6 to 8, wherein the UV light has a wavelength, intensity and pulse duration sufficient to inactivate microorganisms and/or viruses on the surface.

10. The method of any one of claims 6 to 9, wherein the microorganisms and/or viruses comprise SARS-CoV-2.

11. The method of any one of claims 6 to 10, wherein there is a 2 log 10 reduction in the number of microorganisms and viruses on the surface.

12. The method of any one of claims 1 to 11, wherein the UV light irradiates the surface for a period of from 5 ns to 35 ns.

13. The method of any one of claims 1 to 12, wherein the wavelength is 193 nm or 248 nm.

14. The method of any one of claims 1 to 13, wherein the intensity is greater than 6,000 W/cm², preferably greater than 500,000 W/cm².

15. The method of any one of claims 1 to 14, wherein the pulse duration is from about 5 to about 35 ns.

16. The method of any one of claims 1 to 15, wherein a single pulse of the UV light has an energy of at least 0.1 mJ/cm², preferably at least 1.0 mJ/cm², more preferably at least 2.5 mJ/cm² on the surface.

17. The method of any one of claims 1 to 16, wherein a single pulse of the UV light has an energy of at least 4.0 mJ/cm² on the surface.

18. The method of any one of claims 1 to 17, wherein the UV light has a focal spot size approximately the same size as the or each object.

19. The method of any one of claims 1 to 18, wherein the method comprises irradiating the or each object with a single pulse of UV light from a single excimer laser.

20. The method of any one of claims 1 to 19, wherein the method comprises irradiating the or each object with two pulses of UV light from a single excimer laser.

21. The method of any one of claims 1 to 20, wherein a single pulse of UV light illuminates the surface of the or each object uniformly or substantially uniformly.

22. The method of any one of claims 1 to 21, wherein the method comprises:
irradiating a first surface of the or each object with a first pulse of UV light from a first excimer laser, and subsequently irradiating a second surface of the or each object with a second pulse of UV light from a second excimer laser.

23. The method of claim 22, where the first surface and the second surface of the or each object partially or completely overlap.

24. The method of claim 22, where the first surface and the second surface of the or each object do not overlap.

25. The method of any one of claims 22 to 24, where the first pulse and the second pulse are delivered sequentially.

26. The method of any one of claims 22 to 24, where the first pulse and the second pulse are delivered simultaneously.

27. The method of any one of claims 1 to 26, wherein the or each object is conveyed through the UV light at a speed of between 1.0 m/s and 8.0 m/s.

28. The method of any one of claims 1 to 27, wherein the UV light is directed towards the or each object with a turning mirror.

29. The method of any one of claims 1 to 28, wherein the UV light is controlled with a beam expansion lens or beam expanding telescope.

30. The method of any one of claims 1 to 29, wherein the excimer laser is fitted with an unstable resonator.

31. The method of any one of claims 1 to 30, wherein presence of the or each object is detected by a trigger system configured to command the excimer laser to emit the UV light.

32. The method of any one of claims 1 to 31, wherein the UV light is polarised using a polarising element.

33. The method of any one of claims 1 to 32, wherein a flipping mirror is used to direct alternate pulses of UV light into two separate beam paths.

34. The method of claim 33, wherein the flipping mirror is a single axis galvanometer mirror or a 2-dimensional galvanometer mirror or mirror set.

35. The method of any one of claims 1 to 34, comprising reflecting part or all of the UV light onto the or each object using a retroreflective mirror.

36. The method of any one of claims 1 to 35, wherein the UV light is split using a beam splitting element.

37. The method of any one of claims 1 to 36, wherein the surface comprises a polymer.

38. The method of any one of claims 1 to 37, wherein the or each object is a banknote.

39. A sanitised object produced by the method of any one of claims 1 to 38.

40. An apparatus for sanitising a surface of one or more objects, comprising:
an excimer laser that emits pulses of UV light; and
a system that conveys the object(s) through the UV light at a speed of greater than 1.0 m/s, optionally between 1.0 m/s and 8.0 m/s;
wherein the UV light has a wavelength, intensity and pulse duration sufficient to reduce a concentration of a drug substance on the surface and/or reduce microorganisms and/or viruses on the surface.

41. The apparatus of claim 40, wherein the system conveys the object(s) through the UV light at a frequency of greater than 5 Hz, such as between 5 Hz and 50 Hz, or greater than 10 Hz, such as between 10 Hz and 50 Hz.

42. The apparatus of claim 40 or claim 41, wherein the drug substance is cocaine.

43. The apparatus of claim 40 or claim 41, wherein the UV light has a wavelength, intensity and pulse duration sufficient to inactivate microorganisms and/or viruses on the surface.

44. The apparatus of any one of claims 40 to 43, wherein a single pulse of the UV light has an energy of at least 4.0 mJ/cm² on the surface.

45. The apparatus of any one of claims 40 to 44, wherein the apparatus further comprises any one or more of the following: a turning mirror for directing the UV light towards the object(s); a beam expansion lens or beam expanding telescope for controlling beam size; a trigger system configured to detect the presence of the object(s) and command the excimer laser to emit pulses of UV light a polarising element for polarising the UV light; a flipping mirror for directing alternate pulses of the laser into two separate beam paths; a retroreflective mirror for reflecting the UV light onto the object(s); or a beam splitting element for splitting the UV light.

46. The apparatus of any one of claims 40 to 45, wherein the excimer laser is fitted with an unstable resonator.

47. The method of any one of claims 1 to 38 utilising the apparatus of any one of claims 40 to 46.

48. The apparatus of any one of claims 40 to 46 when utilised in the method of any one of claims 1 to 38.

49. The method of any one of claims 2 to 5 or 7 to 38 or the apparatus of any one of claims 41 to 48, wherein the frequency is between 10 Hz and 50 Hz.
